(19) United States
(12) Patent Application Publication
(10) Pub. No.: US 2026/0000670 A1
(43) Pub. Date: Jan. 1, 2026

(54) Prevention or mitigation of adverse effects related to recombinant viral vectors

(71) Applicant: Hoffmann-La Roche Inc., Little Falls, NJ (US)

(72) Inventors: Hélène Cécile HAEGEL, Illkirch-Graffenstaden (FR); Rebecca Joséphine Thérèse XICLUNA, Basel (CH)

(73) Assignee: Hoffmann-La Roche Inc., Little Falls, NJ (US)

(21) Appl. No.: 19/052,088

(22) Filed: Feb. 12, 2025

Related U.S. Application Data
(63) Continuation of application No. PCT/EP2023/072361, filed on Aug. 14, 2023.

(30) Foreign Application Priority Data
Aug. 15, 2022 (EP) 22190354.5

(51) Int. Cl. A61K 31/506; C12N 15/86

(52) U.S. Cl. CPC A61K 31/506; C12N 15/86; C12N 2750/14143

(57) ABSTRACT

The present invention relates to the prevention or mitigation of adverse effects related to gene therapy, such as the formation of anti-drug antibodies. Specifically, the invention relates to the prevention or mitigation of such side effects using a tyrosine kinase inhibitor such as dasatinib.

### CROSS REFERENCE TO RELATED APPLICATIONS

This application is a continuation of International Application No. PCT/EP2023/072361 having an International Filing Date of 14 Aug. 2023, claiming priority to application number EP 22190354.5 filed 15 Aug. 2022, each of which is incorporated herein by reference in its entirety.

### SEQUENCE LISTING

This application contains a Sequence Listing which has been submitted electronically in XML format and is hereby incorporated by reference in its entirety. Said XML copy, created on 10 Feb. 2025, is named “P37522-US_SeqListing.xml” and is 8,525 bytes in size.

### FIELD OF THE INVENTION

The present invention relates to the prevention or mitigation of adverse effects related to gene therapy, such as the formation of anti-drug antibodies. Specifically, the invention relates to the prevention or mitigation of such adverse effectsusing a tyrosine kinase inhibitor such as dasatinib.

### BACKGROUND

Recombinant Adeno-Associated Virus (rAAV) or AAV vectors are viral vectors used for in vivo gene therapy, to deliver a therapeutic transgene into target cells. rAAV-mediated gene therapy holds great promise for a large panel of genetic diseases. Recombinant AAV capsids are commonly derived from wild-type AAVs that naturally infect specific cell types, depending of their serotypes. Recombinant AAV-mediated transgene delivery allows for long-term expression of therapeutic proteins (Nathwani, A. C. et al. Long-term safety and efficacy of factor IX gene therapy in hemophilia B. New England Journal of Medicine 371:1994-2004 (2014)). However, rAAV gene therapy can induce immune responses to the viral capsid and in some cases to the transgene product (Ronzitti, G. et al. Human Immune responses to Adeno-Associated Virus (AAV) vectors. Frontiers in Immunology 11:670 (2020); Shirley, J. L. et al. Immune responses to viral gene therapy vectors. Molecular Therapy 28:709-722 (2020)). Innate and adaptive immune responses may lead to cytokine release, complement activation and cytotoxic T cell responses but most commonly to the formation of antibodies to the AAV capsid. This humoral response is characterised by the secretion of anti-AAV IgM and IgG. These antibodies are, most of the time, neutralizing antibodies that prevent any further rAAV treatment. Moreover, they may cause some of the toxicities observed in clinical trials by mediating complement activation. For these reasons, strategies to mitigate antibody formation to rAA Vs are highly needed (Verdera H. C. et al., AAV Vector Immunogenicity in Humans: A Long Journey to Successful Gene Transfer. Molecular Therapy (2020)).

The tyrosine kinase inhibitor dasatinib was identified as a potent compound to switch off cytokine release and T cell activation in mice treated with T-cell bispecific antibodies (Leclercq et al., Journal for Immuno Therapy of Cancer, 2021; 9 (7)).

### DESCRIPTION OF THE INVENTION

The present inventors have found that a tyrosine kinase inhibitor, in particular dasatinib, may be used to prevent the formation of anti-drug antibodies (ADAs) induced by recombinant viral vector-based gene therapies.

Using an in vivo model of recombinant viral vector-based gene therapy, in particular, AAV based delivery of human proteins (hSEAP, hFactorIX) in mice, the inventors assessed the effects of dasatinib on the (undesired) formation of anti-drug antibodies (ADAs) related to the administration of the recombinant viral vector. Mice were intravenously dosed with a first rAAV8 encoding hSEAP and later redosed with a second rAAV8 encoding hFactorIX. Blood samples were collected and analyzed for presence of ADAs and transgene expression. The inventors show that dasatinib can efficiently reduce ADA formation after recombinant AAV (rAAV) administration in vivo. Furthermore, the inventors show that dasatinib allows re-administration of the same serotype of rAAV. These effects can be obtained at dasatinib concentrations that are clinically relevant doses. The inventors propose that co-administration of dasatinib together with a recombinant viral vecor prevents formation of ADAs against the recombinant viral vector. This invention is widely applicable to the enhancement of gene therapy treatments. For example, overcoming ADAs to the AAV capsid has the potential to enable repeat dosing of patients previously administered with an AAV gene therapy product where effective levels have either not been achieved or have been lost due to time or other confounding issue.

Accordingly, in a first aspect, the present invention provides recombinant viral vector comprising a heterologous polynucleotide for use in the treatment of a disease in an individual, wherein said treatment comprises
(a) the administration of the recombinant viral vector to the individual, and
(b) the administration of a tyrosine kinase inhibitor (TKI) to the individual for prevention or reduction of the formation of anti-drug antibodies (ADAs) related to the administration of the recombinant viral vector.

The invention further provides the use of a recombinant viral vector comprising a heterologous polynucleotide in the manufacture of a medicament for the treatment of a disease in an individual, wherein said treatment comprises
(a) the administration of the recombinant viral vector to the individual, and
(b) the administration of a tyrosine kinase inhibitor (TKI) to the individual for prevention or reduction of the formation of anti-drug antibodies (ADAs) related to the administration of the recombinant viral vector.

The invention also provides a method for treatment of a disease in an individual, wherein said method comprises
(a) the administration of a recombinant viral vector comprising a heterologous polynucleotide to the individual, and
(b) the administration of a tyrosine kinase inhibitor (TKI) to the individual for prevention or reduction of the formation of anti-drug antibodies (ADAs) related to the administration of the recombinant viral vector.

In another aspect, the invention provides a tyrosine kinase inhibitor (TKI) for use in the prevention or reduction of the formation of anti-drug antibodies (ADAs) related to the administration of a recombinant viral vector comprising a heterologous polynucleotide to an individual.

The invention further provides the use of a tyrosine kinase inhibitor (TKI) in the manufacture of a medicament for prevention or reduction of the formation of anti-drug antibodies (ADAs) related to the administration of a recombinant viral vector comprising a heterologous polynucleotide to an individual.

The invention also provides a method for preventing or mitigating formation of anti-drug antibodies (ADAs) related to the administration of a recombinant viral vector comprising a heterologous polynucleotide to an individual, comprising the administration of a tyrosine kinase inhibitor (TKI) to the individual.

Terms are used herein as generally used in the art, unless otherwise defined herein.

In some aspects, the TKI is a Lck and/or Src kinase inhibitor. In more specific aspects, the TKI is dasatinib.

“Dasatinib” is a tyrosine kinase inhibitor (TKI). It is sold under the brand name Sprycel® (among others), for the treatment of certain cases of chronic myelogenous leukemia (CML) and acute lymphoblastic leukemia (ALL). Its CAS number, IUPAC name and chemical structure are shown below.

CAS number: 302962-49-8

IUPAC name: N-(2-chloro-6-methylphenyl)-2-[[6-[4-(2-hydroxyethyl)-1-piperazinyl]-2-methyl-4-pyrimidinyl]amino]-5-thiazole carboxamide monohydrate

Chemical structure:

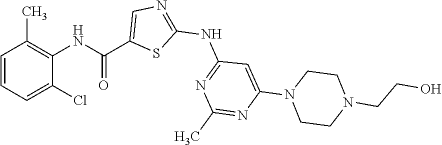 [SMILES: Cc1nc(Nc2ncc(C(=O)Nc3c(C)cccc3Cl)s2)cc(N2CCN(CCO)CC2)n1]

In some aspects, (administration of) the TKI causes inhibition of the formation of anti-drug antibodies (ADAs) that bind to the recombinant viral vector.

An “anti-drug antibody” or “ADA” refers to an antibody that binds to a therapeutic agent (for example to an AAV capsid protein) and may influence serum concentrations and function of the therapeutic agent in an individual. The presence of AD Asmay increase clearance of the therapeutic agent through formation of immune complexes between therapeutic agent and antibody (neutralizing, non-neutralizing or both), thus reducing the therapeutic agent's half-life. Furthermore, the activity and effectiveness of the therapeutic agent (e.g. capacity of transducing target cells) may be decreased through binding of antibody to the therapeutic agent (particularly in the case of neutralizing ADAs). ADAs can also be associated with allergic or hypersensitivity reactions and other adverse events.

“Formation of anti-drug antibodies” or “formation of ADAs” refers toimmunological, in particular humoral, responses in an individual's body triggered by the application of a therapeutic agent (for example an AAV-based recombinant viral vector). Such responses may include cellular response(s) of T cells, particularly CD4+ T cells, such as proliferation, differentiation, cytokine secretion, and/or expression of activation markers. Furthemore, such responses may include activation of B cells, formation of plasma cells, formation of memory B cells and/or excretion of antibodies (such as ADAs).

In some aspects, (administration of) the TKI causes inhibition of the activation of T cells (induced by the recombinant viral vector).

“Activation of T cells” or “T cell activation” as used herein refers to one or more cellular response of a T lymphocyte, particularly a CD4+or CD8+ T cell, selected from: proliferation, differentiation, cytokine secretion, cytotoxic effector molecule release, cytotoxic activity, and expression of activation markers. Suitable assays to measure T cell activation are known in the art and described herein. In particular aspects, T cell activation is determined by measuring expression of CD25 and/or CD69 on the T cell, e.g. by flow cytometry.

In some aspects, (administration of) the TKI causes inhibition of the proliferation of T cells (induced by the recombinant viral vector).

In some aspects, (administration of) the TKI causes inhibition of the cytotoxic activity of T cells (for example directed against a cell expressing a polypeptide or peptide encoded by a heterologous polynucleotide introduced in the cell by a recombinant viral vector).

“Cytotoxic activity” of a T cell refers to the induction of lysis (i.e. killing) of cells by a T lymphocyte, particularly a CD8+ T cell. Cytotoxic activity typically involves degranulation of the T lymphocyte, associated with the release of cytotoxic effector molecules such as granzyme B and/or perforin from the T lymphocyte.

In some aspects, (administration of) the TKI causes inhibition of T cell receptor signaling in T cells (induced by the recombinant viral vector).

By “T cell receptor signaling” is meant activity of the signaling pathway downstream of the T cell receptor (TCR) in a T lymphocyte following engagement of the TCR, involving signaling molecules including tyrosine kinases such as Lck kinase.

In some aspects, (administration of) the TKI causes inhibition of activation of B cells (induced by the recombinant viral vector.

By “activation of B cells” is meant one or more cellular response of a B lymphocyte, particularly a naïve or memory B cell, selected from: proliferation, differentiation (in particular into an antibody-secreting effector cell such as a plasmablast or plasma cell), antibody production, cytokine secretion, and expression of activation and/or differentiation markers. Suitable assays to measure B cell activation are known in the art and described herein. In particular aspects, B cell activation is determined by measuring expression of CD69 on the B cell, e.g. by flow cytometry.

In some aspects, (administration of) the TKI causes inhibition of cytokine secretion by immune cells (induced by the recombinant viral vector). In some aspects, said cytokine is one or more cytokine selected from the group consisting of IL-2, TNF-α, IFN-γ, IL-6 and IL-1β. In some aspects, said immune cells are myeloid cells, CD8+ T cells or CD4+cells.

In some aspects, said inhibition is reversible (i.e. said inhibition can be undone, such that the level of the inhibited parameter returns to about the level it had before the inhibition). In some aspects, said inhibition is reversed after the TKI has not been administered (to the individual) for a given period of time (i.e. after the administration of the TKI is stopped). In some aspects, said period of time is about 1 hour, 2 hours, 3 hours, 4 hours, 5 hours, 6 hours, 7 hours, 8 hours, 12 hours, 16hours, 20 hours, 24 hours, 36 hours, 48 hours, 72 hours, or 96 hours.

Said inhibition may be partial or complete. In some aspects, said inhibition is clinically meaningful and/or statistically significant.

In some aspects, (administration of) the TKI causes reduction of the serum level of one of more cytokine in the individual. In some aspects, said one or more cytokine is selected from the group consisting of IL-2, TNF-α, IFN-γ, IL-6 and IL-1β. In some aspects, said reduction is sustained after the TKI has not been administered (to the individual) for a given amount of time. In some aspects, said amount of time is about 1 hour, 2 hours, 3 hours, 4 hours, 5 hours, 6 hours, 7 hours, 8 hours, 12 hours, 16 hours, 20 hours, 24 hours, 36 hours, 48 hours, 72 hours, or 96 hours. Said reduction of the serum level is in particular as compared to the serum level in an individual (including the same individual) without administration of the TKI (i.e. in such case the serum level is reduced as compared to the serum level without/before administration of the TKI). Said reduction of the serum level is in particular as compared to the serum level in an individual (including the same individual) with administration (in particular first administration) of the recombinant viral vector but without administration of the TKI (i.e. in such case the serum level is reduced as compared to the serum level with/after administration of the recombinant viral vector but without/before administration of the TKI). Without said reduction, the serum level and/or cytokine secretion particularly may be elevated/increased in relation to the (administration of) the recombinant viral vector. In some aspects, said reduction is clinically meaningful and/or statistically significant.

In some aspects, administration of the TKI is upon (clinical) manifestation of increase of the serum level of one of more cytokine. In some aspects, said one or more cytokine is selected from the group consisting of IL-2, TNF-α, IFN-γ, IL-6 and IL-1β. Said administration may be, for example, within about 1 hour, 2 hours, 3 hours, 4 hours, 5 hours, 6 hours, 7 hours, 8 hours, 12 hours, 16 hours, 20 hours, 24 hours, 36 hours, 48 hours, 72 hours, or 96 hours after manifestation of the increase of the serum level of one of more cytokine (i.e. the occurrence clinical symptoms, such as fever). In some aspects, administration of the TKI is in response to the (clinical) manifestation of the increase of the serum level of one of more cytokine (in the individual).

In some aspects, administration of the TKI is before the administration of the recombinant viral vector. In some aspects, administration of the TKI is concurrent to the administration of the recombinant viral vector. In some aspects, administration of the TKI is after the administration of the recombinant viral vector. Where administration of the TKI is before or after the administration of the recombinant viral vector, such administration of the TKI may be, for example, within about 1 hour, 2 hours, 3 hours, 4 hours, 5 hours, 6 hours, 7 hours, 8 hours, 12 hours, 16 hours, 20 hours or 24 hours before or after, respectively, the administration of the recombinant viral vector. Administration of the TKI may be intermittently or continuously. In some aspects, administration of the TKI is oral.

In some aspects, administration of the TKI is at a dose sufficient to cause inhibition of an adverse effect of the recombinant viral vector. An “adverse effect”, which is sometimes also denoted as “side effect” or “adverse event” (especially in clinical studies) is a harmful and undesired effect resulting from medication in the treatment of an individual, herein particularly with a recombinant viral vector. In some aspects, administration of the TKI is at a dose sufficient to cause inhibition of formation of ADAs that bind to the recombinant viral vector. In some aspects, administration of the TKI is at a dose sufficient to cause inhibition of the activation of T cells (induced by the recombinant viral vector). In some aspects, administration of the TKI is at a dose sufficient to cause inhibition of the cytotoxic activity of T cells (induced by the recombinant viral vector). In some aspects, administration of the TKI is at a dose sufficient to cause inhibition of T cell receptor signaling in T cells (induced by the recombinant viral vector). In some aspects, administration of the TKI is at a dose sufficient to cause inhibition of cytokine secretion by immune cells (induced by the recombinant viral vector). In some aspects, said cytokine is one or more cytokine selected from the group consisting of IL-2, TNF-α, IFN-γ, IL-6 and IL-1β. In some aspects, said immune cells are myeloid cells, CD8+ T cells or CD4+ cells. Said inhibition may be partial or complete. In some aspects, said inhibition is clinically meaningful and/or statistically significant.

In some aspects, administration of the TKI is at a dose sufficient to cause inhibition of antibody production (e.g. ADAs) of B cells (induced by the recombinant viral vector). In some aspects, administration of the TKI is at a dose sufficient to cause inhibition of activation of B cells (induced by the recombinant viral vector). In some aspects, administration of the TKI is at a dose sufficient to cause inhibition of differentiation of B cells (induced by the recombinant viral vector). In some aspects, administration of the TKI is at a dose sufficient to cause inhibition of formation of plasma cells (induced by the recombinant viral vector). In some aspects, administration of the TKI is at a dose sufficient to cause sufficient to cause inhibition of cytokine secretion by B cells (induced by the recombinant viral vector). In some aspects, said cytokine is one or more cytokine selected from the group consisting of IL-2, TNF-α, IFN-γ, IL-4, IL-6 and GM-CSF. Said inhibition may be partial or complete. In some aspects, said inhibition is clinically meaningful and/or statistically significant.

Said reduction of the serum level or cytokine secretion is in particular as compared to the serum level or cytokine secretion in an individual (including the same individual) without administration of the TKI (i.e. in such case the serum level is reduced as compared to the serum level without/before administration of the TKI). Said reduction of the serum level or cytokine secretion is in particular as compared to the serum level or cytokine secretion in an individual (including the same individual) with administration (in particular first administration) of the recombinant viral vector but without administration of the TKI (i.e. in such case the serum level is reduced as compared to the serum level with/after administration of the recombinant viral vector but without/before administration of the TKI). Without said reduction, the serum level and/or cytokine secretion particularly may be elevated/increased in relation to the (administration of) the recombinant viral vector. In some aspects, said reduction is clinically meaningful and/or statistically significant. Said inhibition may be partial or complete. In some aspects, said inhibition is clinically meaningful and/or statistically significant.

In some aspects, administration of the TKI is at an effective dose.

An “effective amount” or “effective dose” of an agent, e.g. a TKI or a viral vector, refers to an amount effective, at dosages and for periods of time necessary, to achieve the desired therapeutic or prophylactic result.

In some aspects, administration of the TKI is at a dose of about 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg. In some aspects, administration of the TKI is at a dose of about 20 mg. In some aspects, administration of the TKI is at a dose of about 70 mg. In some aspects, administration of the TKI is at a dose of about 80 mg. In some aspects, administration of the TKI is at a dose of about 100 mg. In some aspects, administration of the TKI is at a dose of about 140 mg.

In some aspects, administration of the TKI is at a dose of about 100 mg or lower. In some aspects, administration of the TKI is at a dose of about 20 mg. In some aspects, administration of the TKI is at a dose of about 70 mg. In some aspects, administration of the TKI is at a dose of about 80 mg. In some aspects, administration of the TKI is at a dose of about 100 mg.

In some aspects, administration of the TKI is daily. In some aspects, administration of the TKI is once daily. In some aspects, administration of the TKI is once daily at a dose of about 100 mg. In some aspects, administration of the TKI is for the period of time during which the adverse effect persists (i.e. administration of the TKI is from manifestation of the adverse effect until reduction or disappearance of the adverse effect). In some aspects, administration of the TKI is stopped after prevention or reduction of formation of ADAs. In some aspects, administration of the TKI is stopped after reduction or ADAs. Said reduction particularly is clinically meaningful and/or statistically significant. In some aspects, administration of the TKI is once, twice, three times, four times, five times, six times, seven times, eight times, nine times or ten times, particularly once, twice, three times, four times, five times, six times, seven times, eight times, nine times or ten times in the course of the treatment of the individual with the recombinant viral vector. In some aspects, administration of the TKI is for 1 day, 2 days, 3 days, 4 days, 5 days, 6 days, 7 days, 8 days, 9 days or 10 days. In some aspects, administration of the TKI is once daily for 1 day, 2 days, 3 days, 4 days, 5 days, 6 days, 7 days, 8 days, 9 days or 10 days. In some aspects, administration of the TKI is associated with the first administration of the recombinant viral vector. Said first administration is particularly the first administration of the recombinant viral vector in the course of the treatment of the individual with the recombinant viral vector. In some aspects, administration of the TKI is concurrent with the first administration of the recombinant viral vector. In some aspects, administration of the TKI is prior to the first administration of the recombinant viral vector. In some aspects, administration of the TKI is subsequent to the first administration of the recombinant viral vector. In some aspects, administration of the TKI is subsequent to the first administration of the recombinant viral vector and prior to a second administration of the recombinant viral vector. Where administration of the TKI is prior or subsequent to the (first) administration of the recombinant viral vector, such administration of the TKI may be, for example, within about 1 hour, 2 hours, 3 hours, 4 hours, 5 hours, 6 hours, 7 hours, 8 hours, 12 hours, 16 hours, 20 hours, 24 hours, 36 hours, 48 hours, 72 hours, or 96 hours before or after, respectively, the administration of the recombinant viral vector.

In some aspects, the administration of the recombinant viral vector is a single administration or a repeated administration. In the course of the treatment of the individual with the recombinant viral vector, the recombinant viral vector may be administered once or several times. In some aspects, the administration of the recombinant viral vector comprises a first and a second administration.

In certain embodiments, (recombinant) viral vectors that may be used in the invention include, for example and without limitation, AAV particles. In certain embodiments, viral vectors that may be used in the invention include, for example and without limitation, retroviral, adenoviral, helper-dependent adenoviral, hybrid adenoviral, herpes simplex virus, lentiviral, poxvirus, Epstein-Barr virus, vaccinia virus, and human cytomegalovirus vectors, including recombinant versions thereof. In a preferred embodiment, recombinant viral vector comprises a lentiviral vector, an adenoviral vector or an adeno-associated (AAV) vector.

The term “recombinant”, as a modifier of a viral vector, such as a recombinant AAV (rAAV) vector, as well as a modifier of sequences such as recombinant polynucleotides and polypeptides, means that compositions have been manipulated (i.e., engineered) in a fashion that generally does not occur in nature. A particular example of a recombinant AAV vector would be where a nucleic acid that is not normally present in a wild-type AAV genome (heterologous polynucleotide) is inserted within a viral genome. An example of which would be where a nucleic acid (e.g., gene) encoding a therapeutic protein or polynucleotide sequence is cloned into a vector, with or without 5′, 3′ and/or intron regions that the gene is normally associated within the AAV genome. Although the term “recombinant” is not always used herein in reference to an AAV vector, as well as sequences such as polynucleotides, recombinant forms including AAV vectors, polynucleotides, etc., are expressly included in spite of any such omission.

A “rAAV vector,” for example, is derived from a wild-type genome of AAV by using molecular methods to remove all or a part of a wild-type AAV genome, and replacing with a non-native (heterologous) nucleic acid, such as a nucleic acid encoding a therapeutic protein or polynucleotide sequence. Typically, for a rAAV vector one or both inverted terminal repeat (ITR) sequences of AAV genome are retained. A rAAVis distinguished from an AAV genome since all or a part of an AAV genome has been replaced with a non-native sequence with respect to the AAV genomic nucleic acid, such as with a heterologous nucleic acid encoding a therapeutic protein or polynucleotide sequence. Incorporation of a non-native (heterologous) sequence therefore defines an AAV as a “recombinant” AAV vector, which can be referred to as a “rAAV vector.”

A recombinant AAV vector sequence can be packaged, referred to herein as a “particle” for subsequent infection (transduction) of a cell, ex vivo, in vitro or in vivo. Where a recombinant vector sequence is encapsidated or packaged into an AAV particle, the particle can also be referred to as a “rAAV,” “rAAV particle” and/or “rAAV virion”. Such rAAV, rAAV particles and rAAV virions include proteins that encapsidate or package a vector genome. Particular examples include in the case of AAV, capsid proteins.

A “vector genome”, which may be abbreviated as “vg”, refers to the portion of the recombinant plasmid sequence that is ultimately packaged or encapsidated to form a rAAV particle. In cases where recombinant plasmids are used to construct or manufacture recombinant AAV vectors, the AAV vector genome does not include the portion of the “plasmid” that does not correspond to the vector genome sequence of the recombinant plasmid. This non-vector genome portion of the recombinant plasmid is referred to as the “plasmid backbone”, which is important for cloning and amplification of the plasmid, a process that is needed for propagation and recombinant AAV vector production, but is not itself packaged or encapsidated into rAAV particles. Thus, a “vector genome” refers to the nucleic acid that is packaged or encapsidated by rAAV.

As used herein, the term “serotype” in reference to an AAV vector means a capsid that is serologically distinct from other AAV serotypes. Serologic distinctiveness is determined on the basis of lack of cross-reactivity between antibodies to one AAV as compared to another AAV. Cross-reactivity differences are usually due to differences in capsid protein sequences/antigenic determinants (e.g., due to VP1, VP2, and/or VP3 sequence differences of AAV serotypes). An antibody to one AAV may cross-react with one or more other AAV serotypes due to homology of capsid protein sequence.

Under the traditional definition, a serotype means that the virus of interest has been tested against serum specific for all existing and characterized serotypes for neutralizing activity and no antibodies have been found that neutralize the virus of interest. As more naturally occurring virus isolates are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

rAAV viral vectors include any viral strain or serotype. For example and without limitation, a rAAV vector genome or particle (capsid, such as VP1, VP2 and/or VP3) can be based upon any AAV serotype, such as AAV-1, -2, -3, -4, -5, -6, -7, -8, -9, -10, -11, -12, -rh74, -rhIO, AAV3B or AAV-2i8, for example. Such vectors can be based on the same strain or serotype (or subgroup or variant), or be different from each other. For example and without limitation, a rAAV plasmid or vector genome or particle (capsid) based upon one serotype genome can be identical to one or more of the capsid proteins that package the vector. In addition, a rAAV plasmid or vector genome can be based upon an AAV serotype genome distinct from one or more of the capsid proteins that package the vector genome, in which case at least one of the three capsid proteins could be a different AAV serotype, e.g., AAV1, AAV2, AAV3, AAV4, AAV5, AAV6, AAV7, AAV8, AAV9, AAV10, AAV11, AAV12, -rh74, -rhIO, AAV3B, AAV-218, or variant thereof, for example. More specifically, a rAAV2 vector genome can comprise AAV2 ITRs but capsids from a different serotype, such as AAV1, AAV3, AAV4, AAV5, AAV6, AAV7, AAV8, AAV9, AAV10, AAV11, AAV12, -rh74, -rhIO, AAV3B, AAV-218, or variant thereof, for example. Accordingly, rAAV vectors include gene/protein sequences identical to gene/protein sequences characteristic for a particular serotype, as well as “mixed” serotypes, which also can be referred to as “pseudotypes.”

In certain embodiments, the rAAV plasmid or vector genome or particle is based upon reptile or invertebrate AAV variants, such as snake and lizard parvovirus (Penzes et al., 2015, J. Gen. Virol., 96:2769-2779) or insect and shrimp parvovirus (Roekring et al., 2002, Virus Res., 87:79-87).

In certain embodiments, the recombinant plasmid or vector genome or particle is based upon a bocavirus variant. Human bocavirus variants are described, for example, in Guido et al., 2016, World J. Gastroenterol., 22:8684-8697.

In one embodiment, the recombinant viral vector comprises proteins to which the ADAs bind. In one embodiment, the recombinant lentiviral vector comprises envelope proteins to which the ADAs bind. In one embodiment, the recombinant AAV (rAAV) vector comprises capsid proteins to which the ADAs bind.

In one embodiment, the recombinant AAV (rAAV) vector comprises VP1, VP2, and/or VP3 capsid protein having 70% or more sequence identity to VP1, VP2 and/or VP3 capsid protein selected from the group consisting of AAV1, AAV2, AAV3, AAV4, AAV5, AAV6, AAV7, AAV8, AAV9, AAV10, AAV11, AAV12, -rh74, -rhIO, AAV3B, AAV-218 VP1, VP2 and/or VP3 capsid protein. In one embodiment, the recombinant AAV (rAAV) vector comprises VP1, VP2, and/or VP3 capsid protein having 100% sequence identity to VP1, VP2 and/or VP3 capsid protein selected from the group consisting of AAV1, AAV2, AAV3, AAV4, AAV5, AAV6, AAV7, AAV8, AAV9, AAV 10, AAV11, AAV12, -rh74, -rh10, AAV3B, AAV-218 VP1, VP2 and/or VP3 capsid protein. In certain embodiments, the AAV vector includes or consists of a sequence at least 70% or more (e.g., 75%, 80%, 85%, 90%, 95%, 96%, 97%, 98%, 99%, 99.5%, etc.) identical to one or more AAV1, AAV2, AAV3, AAV4, AAV5, AAV6, AAV7, AAV8, AAV9, AAV10, AAV11, AAV12, -rh74, -rhlO or AAV3B, ITR(s).

In certain embodiments, the recombinant AAV (rAAV) vectors include AAV1, AAV2, AAV3, AAV4, AAV5, AAV6, AAV7, AAV8, AAV9, AAV10, AAV11, AAV12, AAV3B, RhIO, Rh74 and AAV-218 variants (e.g., ITR and capsid variants, such as amino acid insertions, additions, substitutions and deletions) thereof, for example, as set forth in WO 2013/158879 (International Application PCT/US2013/037170), WO 2015/013313 (International Application PCT/US2014/047670) and US 2013/0059732 (U.S. application Ser. No. 13/594,773).

In a preferred embodiment, the recombinant viral vector is selected from the group consisting of AAV2, AAV8 and AAV9. In one such preferred embodiment, the recombinant AAV (rAAV) vector is selected from the group consisting of rAAV2, rAAV8 and rAAV9. In one such preferred embodiment, the AAV vector comprises VP1, VP2, and/or VP3 capsid protein having 70% or more sequence identity to VP1, VP2 and/or VP3 capsid protein selected from the group consisting of AAV2, AAV8 and AAV8 VP1, VP2 and/or VP3 capsid protein. In one such preferred embodiment, the AAV vector comprises VP1, VP2, and/or VP3 capsid protein having 100% or more sequence identity to VP1, VP2 and/or VP3 capsid protein selected from the group consisting of AAV2, AAV8 and AAV8 VP1, VP2 and/or VP3 capsid protein.

24. The recombinant viral vector, TKI, use or method of claim 19, 21 or 22, wherein the AAV vector comprises VP1, VP2, and/or VP3 capsid protein having 100% sequence identity to VP1, VP2 and/or VP3 capsid protein selected from the group consisting of AAV1, AAV2, AAV3, AAV4, AAV5, AAV6, AAV7, AAV8, AAV9, AAV 10, AAV11, AAV12, -rh74, -rhIO, AAV3B, AAV-218 VP1, VP2 and/or VP3 capsid protein rAAV, such as AAV1, AAV2, AAV3, AAV4, AAV5, AAV6, AAV7, AAV8, AAV9, AAV 10, AAV11, AAV12, -rh74, -rh10, AAV3B, AAV-218 and variants, hybrids and chimeric sequences, can be constructed using recombinant techniques that are known to a skilled artisan, to include one or more heterologous polynucleotide sequences (transgenes) flanked with one or more functional AAV ITR sequences. Such AAV vectors typically retain at least one functional flanking ITR sequence(s), as necessary for the rescue, replication, and packaging of the recombinant vector into a rAAV vector particle. A rAAV vector genome would therefore include sequences required in cis for replication and packaging (e.g., functional ITR sequences).

In certain embodiments, a lenti virus used in the invention may be a human immunodeficiency-1 (HIV-1), human immunodeficiency-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), or caprine arthritis encephalitis virus (CAEV). Lentiviral vectors are capable of providing efficient delivery, integration and long-term expression of heterologous polynucleotide sequences into non-dividing cells both in vitro and in vivo. A variety of lentiviral vectors are known in the art, see Naldini et al. (Proc. Natl. Acad. Sci. USA, 93:11382-11388 (1996); Science, 272:263-267 (1996)), Zufferey et al., (Nat. Biotechnol, 15:871-875, 1997), Dull et al, (J Virol. 1998 November;72(ll): 8463-71, 1998), U.S. Pat. Nos. 6,013,516 and 5,994,136, any of which may be a suitable viral vector for use in the invention.

An immune response, such as humoral immunity, can also develop against a recombinant viral vector, and/or a heterologous polynucleotide or a protein or peptide encoded by a heterologous polynucleotide encapsidated by the viral vector, resulting in inhibition or reduction in viral vector cell transduction, heterologous polynucleotide expression or function, or function or activity of the protein or peptide encoded by a heterologous polynucleotide in a subject to which the viral vector is administered.

Antibodies (such as ADAs) that bind to a viral vector used in the invention, such as a recombinant viral vector, which can be referred to as “neutralizing” antibodies, can reduce or inhibit cell transduction of viral vectors useful for gene therapy. As a result, while not being bound by theory, cell transduction is reduced or inhibited thereby reducing introduction of the viral packaged heterologous polynucleotide into cells and subsequent expression and, as appropriate, subsequent translation into a protein or peptide. Additionally, antibodies that bind to a heterologous polynucleotide or a protein or peptide encoded by a heterologous polynucleotide encapsidated by the viral vector can inhibit expression of a heterologous polynucleotide, function or activity of a heterologous polynucleotide or function or activity of a protein or peptide encoded by a heterologous polynucleotide.

Accordingly, antibodies (such as ADAs) can be present that bind to a recombinant viral vector (e.g., AAV) and/or antibodies can be present that bind to a protein or peptide encoded by a heterologous polynucleotide in a subject. In addition, antibodies can be present that bind to a heterologous polynucleotide encapsidated by the recombinant viral vector.

Antibodies that bind to a recombinant viral vector (e.g., AAV) or that bind to a protein or peptide encoded by a heterologous polynucleotide, should they be induced, can be reduced or abolished in a subject by use of a tyrosine kinase inhibitor (TKI) as set forth herein.

In one embodiment, the ADAs comprise IgG, IgM, IgA, IgD and/or IgE. IgG, IgM, IgA, IgD and/or IgE antibodies that bind to a recombinant viral vector (e.g., rAAV) or that bind to a protein or polypeptide encoded by a heterologous polynucleotide, should they be induced, can be reduced or abolished in a subject by use of a tyrosine kinase inhibitor (TKI) as set forth herein. Reduction of circulating antibodies (e.g. reduced levels of antibodies in blood, plasma or serum), can be measured by standard assays known in the art and as herein described. In one embodiment, the ADAs comprise IgG and/or IgG.

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids include genomic DNA, cDNA and antisense DNA, and spliced or unspliced mRNA, IRNA tRNA and inhibitory DNA or RNA (RNAi, e.g., small or short hairpin (sh) RNA, microRNA (miRNA), small or short interfering (si) RNA, trans-splicing RNA, or antisense RNA).

Nucleic acids include naturally occurring, synthetic, and intentionally modified or altered polynucleotides. Nucleic acids can be single, double, or triplex, linear or circular, and can be of any length. In discussing nucleic acids, a sequence or structure of a particular polynucleotide may be described herein according to the convention of providing the sequence in the 5′ to 3′ direction.

A “heterologous” polynucleotide or nucleic acid sequence refers to a polynucleotide inserted into a plasmid or vector for purposes of vector mediated transfer/delivery of the polynucleotide into a cell. Heterologous nucleic acid sequences are distinct from viral nucleic acid, i.e., are non-native with respect to viral nucleic acid. Once transferred/delivered into the cell, a heterologous nucleic acid sequence, contained within the vector, can be expressed (e.g., transcribed, and translated if appropriate). Alternatively, a transferred/delivered heterologous polynucleotide in a cell, contained within the vector, need not be expressed. Although the term “heterologous” is not always used herein in reference to nucleic acid sequences and polynucleotides, reference to a nucleic acid sequence or polynucleotide even in the absence of the modifier “heterologous” is intended to include heterologous nucleic acid sequences and polynucleotides in spite of the omission.

A “transgene” is used herein to refer to a nucleic acid that is intended or has been introduced into a cell or organism. Transgenes include any nucleic acid, such as a heterologous polynucleotide sequence or a heterologous nucleic acid encoding a protein or peptide. The term transgene and heterologous nucleic acid/polynucleotide sequences are used interchangeably herein.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

In one embodiment, the individual has a lung disease (e.g., cystic fibrosis), a bleeding disorder (e.g., hemophilia A or hemophilia B with or without inhibitors), thalassemia, a blood disorder (e.g., anemia), Alzheimer's disease, Parkinson's disease, Huntington's disease, amyotrophic lateral sclerosis (ALS), epilepsy, a lysosomal storage disease (e.g., aspartylglucosaminuria, Batten disease, late infantile neuronal ceroid lipofuscinosis type 2 (CLN2), cystinosis, Fabry disease, Gaucher disease types I, II, and III, glycogen storage disease II (Pompe disease), GM2-gangliosidosis type I (Tay Sachs disease), GM2-gangliosidosis type II (Sandhoff disease), mucolipidosis types I (sialidosis type I and II), II (I-cell disease), III (pseudo-Hurler disease) and IV, mucopolysaccharide storage diseases (Hurler disease and variants, Hunter, Sanfilippo Types A,B,C,D, Morquio Types A and B, Maroteaux-Lamy and Sly diseases), Niemann-Pick disease types A/B, C1 and C2, and Schindler disease types I and II), hereditary angioedema (HAE), a copper or iron accumulation disorder (e.g., Wilson's or Menkes disease), lysosomal acid lipase deficiency, a neurological or neurodegenerative disorder, cancer, type 1 or type 2 diabetes, adenosine deaminase deficiency, a metabolic defect (e.g., glycogen storage diseases), a disease of solid organs (e.g., brain, liver, kidney, heart), or an infectious viral (e.g., hepatitis B and C, HIV, etc.), bacterial or fungal disease.

In one embodiment, the individual has a blood clotting disorder. In one embodiment, the individual has hemophilia A, hemophilia A with inhibitory antibodies, hemophilia B, hemophilia B with inhibitory antibodies, a deficiency in any coagulation Factor: VII, VIII, IX, X, XI, V, XII, II, von Willebrand factor, or a combined FV/FVIII deficiency, thalassemia, vitamin K epoxide reductase C1 deficiency or gamma-carboxylase deficiency.

In one embodiment, theindividual has anemia, bleeding associated with trauma, injury, thrombosis, thrombocytopenia, stroke, coagulopathy, disseminated intravascular coagulation (DIC); over-anticoagulation associated with heparin, low molecular weight heparin, pentasaccharide, warfarin, small molecule antithrombotics (i.e., FXa inhibitors), or a platelet disorder such as, Bernard Soulier syndrome, Glanzmann thrombasthenia, or storage pool deficiency.

In one embodiment, the individual has a disease that affects or originates in the central nervous system (CNS). In one embodiment, the disease is a neurodegenerative disease. In one embodiment, the CNS or neurodegenerative disease is Alzheimer's disease, Huntington's disease, AFS, hereditary spastic hemiplegia, primary lateral sclerosis, spinal muscular atrophy, Kennedy's disease, a polyglutamine repeat disease, or Parkinson's disease. In one embodiment, the CNS or neurodegenerative disease is a polyglutamine repeat disease. In one embodiment, the polyglutamine repeat disease is a spinocerebellar ataxia (SCA1, SCA2, SCA3, SCA6, SCA7, or SCA17).

In certain embodiments, a heterologous polynucleotide encodes a protein selected from the group consisting of GAA (acid alpha-glucosidase) for treatment of Pompe disease; ATP7B (copper transporting ATPase2) for treatment of Wilson's disease; alpha galactosidase for treatment of Fabry's disease; ASS1 (arginosuccinate synthase) for treatment of Citrullinemia Type 1; beta-glucocerebrosidase for treatment of Gaucher disease Type 1; beta-hexosaminidase A for treatment of Tay Sachs disease; SERPING1 (C1 protease inhibitor or C1 esterase inhibitor) for treatment of hereditary angioedema (HAE), also known as C1 inhibitor deficiency type I and type II; and glucose-6-phosphatase for treatment of glycogen storage disease type I (GSDI).

In certain embodiments, a heterologous polynucleotide encodes a protein selected from the group consisting of insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor a (TGFa), platelet-derived growth factor (PDGF), insulin growth factors I and II (IGF-I and IGF-II), TGFβ, activins, inhibins, bone morphogenic protein (BMP), nerve growth factor (NGF), brain-derived neurotrophic factor (BDNF), neurotrophins NT-3 and NT4/5, ciliary neurotrophic factor (CNTF), glial cell line derived neurotrophic factor (GDNF), neurturin, agrin, netrin-1 and netrin-2, hepatocyte growth factor (HGF), ephrins, noggin, sonic hedgehog and tyrosine hydroxylase.

In certain embodiments, a heterologous polynucleotide encodes acid a-glucosidase (GAA). Administration of a recombinant viral vector comprising a heterologous polynucleotide encoding GAA to a subject with Pompe or another glycogen storage disease can lead to the expression of the GAA protein. Expression of GAA protein in the patient may serve to suppress, inhibit or reduce the accumulation of glycogen, prevent the accumulation of glycogen or degrade glycogen, which in turn can reduce or decrease one or more adverse effects of Pompe disease, or another glycogen storage disease.

In certain embodiments, a heterologous polynucleotide encodes a protein selected from the group consisting of thrombopoietin (TPO), an interleukin (IL-1 through IL-36, etc.), monocyte chemoattractant protein, leukemia inhibitory factor, granulocyte-macrophage colony stimulating factor, Fas ligand, tumor necrosis factors a and b, interferons α, β, and γ, stem cell factor, flk-2/flt3ligand, IgG, IgM, IgA, IgD and IgE, chimeric immunoglobulins, humanized antibodies, single chain antibodies, T cell receptors, chimeric T cell receptors, single chain T cell receptors, class I and class II MHC molecules.

In certain embodiments, a heterologous polynucleotide encodes CFTR (cystic fibrosis transmembrane regulator protein), a blood coagulation (clotting) factor (Factor XIII, Factor IX, Factor VIII, Factor X, Factor VII, Factor Vila, protein C, etc.) a gain of function blood coagulation factor, an antibody, retinal pigment epithelium-specific 65 kDa protein (RPE65), erythropoietin, LDL receptor, lipoprotein lipase, ornithine transcarbamylase, β-globin, a-globin, spectrin, a-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor, epidermal growth factor, nerve growth factor, neurotrophic factor-3 and-4, brain-derived neurotrophic factor, glial derived growth factor, transforming growth factor a and β, a cytokine, a-interferon, β-interferon, interferon-g, interleukin-2, interleukin-4, interleukin 12, granulocyte-macrophage colony stimulating factor, lymphotoxin, a suicide gene product, herpes simplex virus thymidine kinase, cytosine deaminase, diphtheria toxin, cytochrome P450, deoxycytidine kinase, tumor necrosis factor, a drug resistance protein, a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitope or hCDR1, insulin, glucokinase, guanylate cyclase 2D (LCA-GUCY2D), Rab escort protein 1 (Choroideremia), LCA 5 (LCA-Lebercilin), ornithine ketoacid aminotransferase (Gyrate Atrophy), Retinoschisin 1 (X-linked Retinoschisis), USH1C (Usher's Syndrome 1C), X-linked retinitis pigmentosa GTPase (XLRP), MERTK (AR forms of RP: retinitis pigmentosa), DFNB1(Connexin 26 deafness), ACHM 2, 3 and 4 (Achromatopsia), PKD-1 or PKD-2 (Polycystic kidney disease), TPP1, CLN2, a sulfatase, N-acetylglucosamine-1-phosphate transferase, cathepsin A, GM2-AP, NPC1, VPC2, a sphingolipid activator protein, one or more zinc finger nucleases for genome editing, or one or more donor sequences used as repair templates for genome editing.

In certain embodiments, a heterologous polynucleotide encodes erythropoietin (EPO) for treatment of anemia; interferon-alpha, interferon-beta, and interferon-gamma for treatment of various immune disorders, viral infections and cancer; an interleukin (IL), including any one of IL-1 through IL-36, and corresponding receptors, for treatment of various inflammatory diseases or immuno-deficiencies; a chemokine, including chemokine (C-X-C motif) ligand 5 (CXCL5) for treatment of immune disorders; granulocyte-colony stimulating factor (G-CSF) for treatment of immune disorders such as Crohn's disease; granulocyte-macrophage colony stimulating factor (GM-CSF) for treatment of various human inflammatory diseases; macrophage colony stimulating factor (M-CSF) for treatment of various human inflammatory diseases; keratinocyte growth factor (KGF) for treatment of epithelial tissue damage; chemokines such as monocyte chemoattractant protein-1 (MCP-1) for treatment of recurrent miscarriage, HIV-related complications, and insulin resistance; tumor necrosis factor (TNF) and receptors for treatment of various immune disorders; alphal-antitrypsin for treatment of emphysema or chronic obstructive pulmonary disease (COPD); alpha-L-iduronidase for treatment of mucopolysaccharidosis I (MPS I); ornithine transcarbamoylase (OTC) for treatment of OTC deficiency; phenylalanine hydroxylase (PAH) or phenylalanine ammonia-lyase (PAL) for treatment of phenylketonuria (PKU); lipoprotein lipase for treatment of lipoprotein lipase deficiency; apolipoproteins for treatment of apolipoprotein (Apo) A-I deficiency; low-density lipoprotein receptor (LDL-R) for treatment of familial hypercholesterolemia (FH); albumin for treatment of hypoalbuminemia; lecithin cholesterol acyltransferase (LCAT); carbamoyl synthetase I; argininosuccinate synthetase; argininosuccinate lyase; arginase; fumarylacetoacetate hydrolase; porphobilinogen deaminase; cystathionine beta-synthase for treatment of homocystinuria; branched chain ketoacid decarboxylase; isovaleryl-CoA dehydrogenase; propionyl CoA carboxylase; methylmalonyl-CoA mutase; glutaryl CoA dehydrogenase; insulin; pyruvate carboxylase; hepatic phosphorylase; phosphorylase kinase; glycine decarboxylase; H-protein; T-protein; cystic fibrosis transmembrane regulator (CFTR); ATP-binding cassette, sub-family A (ABC1), member 4 (ABCA4) for the treatment of Stargardt disease; or dystrophin.

The terms “polypeptides”, “proteins” and “peptides” are used interchangeably herein. The “polypeptides”, “proteins” and “peptides” encoded by the “polynucleotide sequences”, include full-length native sequences, as with naturally occurring proteins, as well as functional subsequences, modified forms or sequence variants so long as the subsequence, modified form or variant retains some degree of functionality of the native full-length protein. In the invention, such polypeptides, proteins and peptides encoded by the polynucleotide sequences can be but are not required to be identical to the endogenous protein that is defective, or whose expression is insufficient, or deficient in the treated mammal.

In certain embodiments, the heterologous polynucleotide encodes an inhibitory nucleic acid selected from the group consisting of a siRNA, an antisense molecule, miRNA, RNAi, a ribozyme and a shRNA.

In certain embodiments, an inhibitory nucleic acid binds to a gene, a transcript of a gene, or a transcript of a gene associated with a polynucleotide repeat disease selected from the group consisting of a huntingtin (HTT) gene, a gene associated with dentatorubropallidoluysian atrophy (atrophin 1, ATN1), androgen receptor on the X chromosome in spinobulbar muscular atrophy, human Ataxin-1, -2, -3, and -7, Cav2.1 P/Q voltage-dependent calcium channel (CACNA1A), TATA-binding protein, Ataxin 8 opposite strand (ATXN8OS), Serine/threonine-protein phosphatase 2A 55 kDa regulatory subunit B beta isoform in spinocerebellar ataxia (type 1, 2, 3, 6, 7, 8, 12 17), FMR1 (fragile X mental retardation 1) in fragile X syndrome, FMR1 (fragile X mental retardation 1) in fragile X-associated tremor/ataxia syndrome, FMR1 (fragile X mental retardation 2) or AF4/FMR2 family member 2 in fragile XE mental retardation; Myotonin-protein kinase (MT-PK) in myotonic dystrophy; Frataxin in Friedreich's ataxia; a mutant of superoxide dismutase 1 (SOD1) gene in amyotrophic lateral sclerosis; a gene involved in pathogenesis of Parkinson's disease and/or Alzheimer's disease; apolipoprotein B (APOB) and proprotein convertase subtilisin/kexin type 9 (PCSK9), hypercholesterolemia; HIV Tat, human immunodeficiency virus transactivator of transcription gene, in HIV infection; HIV TAR, HIV TAR, human immunodeficiency virus transactivator response element gene, in HIV infection; C-C chemokine receptor (CCR5) in HIV infection; Rous sarcoma virus (RSV) nucleocapsid protein in RSV infection, liver-specific microRNA (miR-122) in hepatitis C virus infection; p53, acute kidney injury or delayed graft function kidney transplant or kidney injury acute renal failure; protein kinase N3 (PKN3) in advance recurrent or metastatic solid malignancies; LMP2, LMP2also known as proteasome subunit beta-type 9 (PSMB 9), metastatic melanoma; LMP7, also known as proteasome subunit beta-type 8 (PSMB 8), metastatic melanoma; MECLI also known as proteasome subunit beta-type 10 (PSMB 10), metastatic melanoma; vascular endothelial growth factor (VEGF) in solid tumors; kinesin spindle protein in solid tumors, apoptosis suppressor B-cell CLL/lymphoma (BCL-2) in chronic myeloid leukemia; ribonucleotide reductase M2 (RRM2) in solid tumors; Furin in solid tumors; polo-like kinase 1 (PLK1) in liver tumors, diacylglycerol acyltransferase 1 (DGAT1) in hepatitis C infection, beta-catenin in familial adenomatous polyposis; beta2 adrenergic receptor, glaucoma; RTP801/Redd 1 also known as DNA damage-inducible transcript 4 protein, in diabetic macular edema (DME) or age-related macular degeneration; vascular endothelial growth factor receptor I (VEGFR1) in age-related macular degeneration or choroidal neovascularization, caspase 2 in non-arteritic ischaemic optic neuropathy; Keratin 6A N17K mutant protein in pachyonychia congenital; influenza A virus genome/gene sequences in influenza infection; severe acute respiratory syndrome (SARS) coronavirus genome/gene sequences in SARS infection; respiratory syncytial virus genome/gene sequences in respiratory syncytial virus infection; Ebola filovirus genome/gene sequence in Ebola infection; hepatitis B and C virus genome/gene sequences in hepatitis B and C infection; herpes simplex virus (HSV) genome/gene sequences in HSV infection, coxsackievirus B3 genome/gene sequences in coxsackievirus B3 infection; silencing of a pathogenic allele of a gene (allele-specific silencing) like torsin A (TORI A) in primary dystonia, pan-class I and HLA-allele specific in transplant; and mutant rhodopsin gene (RHO) in autosomal dominantly inherited retinitis pigmentosa (adRP).

In one embodiment, the protein encoded by the heterologous polynucleotide comprises a gene editing nuclease. In one embodiment, the gene editing nuclease comprises a zinc finger nuclease (ZFN) or a transcription activator-like effector nuclease (TALEN). In one embodiment, the gene editing nuclease comprises a functional Type II CRISPR-Cas9.

For use in the present invention, the recombinant viral vector would be formulated, dosed, and administered in a fashion consistent with good medical practice. Factors for consideration in this context include the particular disorder being treated, the particular mammal being treated, the clinical condition of the individual patient, the cause of the disorder, the site of delivery of the recombinant viral vector, the method of administration, the scheduling of administration, and other factors known to medical practitioners.

An effective amount of the recombinant viral vector may be administered for prevention or treatment of disease. The appropriate route of administration and dosage of the recombinant viral vector may be determined based on the type of disease to be treated, the type of the recombinant viral vector, the severity and course of the disease, the clinical condition of the individual, the individual's clinical history and response to the treatment, and the discretion of the attending physician. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Recombinant viral vectors can be administered at any appropriate dose. Generally, doses will range from at least 1×10⁸, or more, for example, 1×10⁹, 1×10¹⁰, 1×10¹¹, 1×10¹², 1×10¹³ or 1×10¹⁴, or more, vector genomes per kilogram (vg/kg) of the weight of the subject, to achieve a therapeutic effect. AAV dose in the range of 1×10¹⁰-1×10¹¹ vg/kg in mice, and 1×10¹²-1×10¹³ vg/kg in dogs have been effective. More particularly, a dose from about 1×10¹¹ vg/kg to about 5×10¹⁴ vg/kg inclusive, or from about 5×10¹¹ vg/kg to about 1×10¹⁴ vg/kg inclusive, or from about 5×10¹¹ vg/kg to about 5×10¹³ vg/kg inclusive, or from about 5×10¹¹ vg/kg to about 1×10¹³ vg/kg inclusive, or from about 5×10¹¹ vg/kg or about 5×10¹² vg/kg inclusive, or from about 5×10¹¹ vg/kg to about 1×10¹² vg/kg inclusive. Doses can be, for example, about 5×10¹⁴ vg/kg, or less than about 5×10¹⁴ vg/kg, such as a dose from about 2×10¹¹ to about 2×10¹⁴ vg/kg inclusive, in particular, for example, about 2×10¹² vg/kg, about 6×10¹² vg/kg, or about 2×10¹³ vg/kg.

Doses can vary and depend upon the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

The dose to achieve a therapeutic effect, e.g., the dose in vector genomes/per kilogram of body weight (vg/kg), will vary based on several factors including, but not limited to: route of administration, the level of heterologous polynucleotide expression required to achieve a therapeutic effect, the specific disease treated, any host immune response to the recombinant viral vector, a host immune response to the heterologous polynucleotide or expression product (protein or peptide or transcribed nucleic acid), and the stability of the protein or peptide expressed or nucleic acid transcribed. One skilled in the art can determine a recombinant viral vector genome dose range to treat a patient having a particular disease or disorder based on the aforementioned factors, as well as other factors.

An “effective amount” or “sufficient amount” refers to an amount that provides, in single or multiple doses, alone or in combination, with one or more other compositions, treatments, protocols, or therapeutic regimens agents, a detectable response of any duration of time (long or short term), an expected or desired outcome in or a benefit to a subject of any measurable or detectable degree or for any duration of time (e.g., for minutes, hours, days, months, years, or cured). The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is a satisfactory outcome.

The recombinant viral vector and the TKI can be administered by any suitable route, and may be administered by the same route of administration or by different routes of administration. In some aspects, the administration of the recombinant viral vector is parenteral, particularly intravenous.

In some aspects, the administration of the recombinant viral vector is the first administration of the recombinant viral vector to the individual, particularly the first administration of the recombinant viral vector in the course of the treatment of the individual with the recombinant viral vector.

An effective amount or a sufficient amount can but need not be provided in a single administration, may require multiple administrations, and, can but need not be, administered alone or in combination with another composition (e.g., agent), treatment, protocol or therapeutic regimen. For example, the amount may be proportionally increased as indicated by the need of the subject, type, status and severity of the disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered effective also include amounts that result in a reduction of the use of another treatment, therapeutic regimen or protocol, such as administration of recombinant GAA for treatment of a lysosomal storage disease (e.g., Pompe disease), or administration of a recombinant clotting factor protein (e.g., FVIII or FIX) for treatment of a clotting disorder (e.g., hemophilia A (HemA) or hemophilia B (HemB)).

For Pompe disease, an effective amount would be an amount of GAA that inhibits or reduces glycogen production or accumulation, enhances or increases glycogen degradation or removal, reduces lysosomal alterations in tissues of the body of a subject, or improves muscle tone and/or muscle strength and/or respiratory function in a subject, for example. Effective amounts can be determined, for example, by ascertaining the kinetics of GAA uptake by myoblasts from plasma. Myoblasts GAA uptake rates (K uptake) of about 141-147 nM may appear to be effective (see, e.g., Maga et al., J. Biol. Chem. 2012) In animal models, GAA activity levels in plasma of greater than about 1,000 nmol/hr/mL, for example, about 1,000 to about 2,000 nmol/hr/mL have been observed to be therapeutically effective.

For HemA and HemB, generally speaking, it is believed that, in order to achieve a therapeutic effect, a blood coagulation factor concentration that is greater than 1% of factor concentration found in a normal individual is needed to change a severe disease phenotype to a moderate one. A severe phenotype is characterized by joint damage and life-threatening bleeds. To convert a moderate disease phenotype into a mild one, it is believed that a blood coagulation factor concentration greater than 5% of normal is needed.

FVIII and FIX levels in normal humans are about 150-200 ng/ml plasma, but may be less (e.g., range of about 100-150 ng/ml) or greater (e.g., range of about 200-300 ng/ml) and still considered normal, due to functional clotting as determined, for example, by an activated partial thromboplastin time (aPTT) one-stage clotting assay. Thus, a therapeutic effect can be achieved such that the total amount of FVIII or FIX in the subject/human is greater than 1% of the FVIII or FIX present in normal subjects/humans, e.g., 1% of 100-300 ng/mL.

The composition can be administered to a subject as a combination composition, or administered separately, such as concurrently or in series or sequentially (prior to or following) delivery or administration of a recombinant viral vector comprising a heterologous polynucleotide. The invention provides combinations in which a method or use of the invention is in a combination with any compound, agent, drug, therapeutic regimen, treatment protocol, process, remedy or composition, set forth herein or known to one of skill in the art. The compound, agent, drug therapeutic regimen, treatment protocol, process, remedy or composition can be administered or performed prior to, substantially contemporaneously with or following administration of a recombinant viral vector comprising a heterologous polynucleotide, to a subject.

An effective amount or a sufficient amount need not be effective in each and every subject treated, nor a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use.

The term “ameliorate” means a detectable or measurable improvement in a subject's disease or symptom thereof, or an underlying cellular response. A detectable or measurable improvement includes a subjective or objective decrease, reduction, inhibition, suppression, limit or control in the occurrence, frequency, severity, progression, or duration of the disease, or complication caused by or associated with the disease, or an improvement in a symptom or an underlying cause or a consequence of the disease, or a reversal of the disease. For Pompe, an effective amount would be an amount that inhibits or reduces glycogen production or accumulation, enhances or increases glycogen degradation or removal, improves muscle tone and/or muscle strength and/or respiratory function, for example. For HemA or HemB, an effective amount would be an amount that reduces frequency or severity of acute bleeding episodes in a subject, for example, or an amount that reduces clotting time as measured by a clotting assay, for example.

Accordingly, pharmaceutical compositions of the invention include compositions wherein the active ingredients are contained in an effective amount to achieve the intended therapeutic purpose. Determining a therapeutically effective dose is well within the capability of a skilled medical practitioner using techniques and guidance known in the art and using the teachings provided herein.

Therapeutic doses will depend on, among other factors, the age and general condition of the subject, the severity of the aberrant phenotype, and the strength of the control sequences regulating expression levels. Thus, a therapeutically effective amount in humans will fall in a relatively broad range that may be determined by a medical practitioner based on the response of an individual patient to a vector-based treatment. Such doses may be alone or in combination with an immunosuppressive agent or drug.

Compositions such as pharmaceutical compositions may be delivered to a subject, so as to allow transgene expression and optionally production of encoded protein. In certain embodiments, pharmaceutical compositions comprising sufficient genetic material to enable a subject to produce a therapeutically effective amount of a blood-clotting factor to improve hemostasis in the subject. In certain embodiments, pharmaceutical compositions comprising sufficient heterologous polynucleotide to enable a subject to produce a therapeutically effective amount of GAA.

In certain embodiments, a therapeutic effect in a subject is sustained for a period of time, e.g., 2-4, 4-6, 6-8, 8-10, 10-14, 14-20, 20-25, 25-30, or 30-50 days or more, for example, 50-75, 75-100, 100-150, 150-200 days or more. Accordingly, in certain embodiments, a recombinant viral vector provides a therapeutic effect.

An “individual” or “subject” herein is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). In certain aspects, the individual or subject is a human. In some aspects, the individual has a disease, particularly a disease treatable or to be treated by the recombinant viral vector.

In some aspects, the individual has an elevated serum level of one of more cytokine. In some aspects, said elevated serum level is related to the administration of the recombinant viral vector to the individual. Said elevated serum level is in particular as compared to the serum level in a healthy individual, and/or the serum level in an individual (including the same individual) without administration of the recombinant viral vector (i.e. in such case the serum level is elevated as compared to the serum level without administration of the recombinant viral vector). In some aspects, said one or more cytokine is selected from the group consisting of IL-2, TNF-α, IFN-γ, IL-6 and IL-1β.

A cytokine according to any of the aspects of the invention is preferably a proinflammatory cytokine, in particular one or more cytokine selected from the group consisting of IL-2, TNF-α, IFN-γ, IL-6 and IL-1β. In some aspects, the cytokine is IL-2. In some aspects, the cytokine is TNF-α. In some aspects, the cytokine is IFN-γ. In some aspects, the cytokine is IL-6. In some aspects, the cytokine is IL-1β.

In some aspects, the treatment with or administration of the recombinant viral vector may result in a response in the individual. In some aspects, the response may be a complete response. In some aspects, the response may be a sustained response after cessation of the treatment. In some aspects, the response may be a complete response that is sustained after cessation of the treatment. In other aspects, the response may be a partial response. In some aspects, the response may be a partial response that is sustained after cessation of the treatment. In some aspects, the treatment with or administration of the recombinant viral vector and the TKI may improve the response as compared to treatment with or administration of the recombinant viral vector alone (i.e. without the TKI). In some aspects, the treatment or administration of the recombinant viral vector and the TKI may increase response rates in a patient population, as compared to a corresponding patient population treated with the recombinant viral vector alone (i.e. without the TKI).

In one aspect, the individual is at risk of developing ADAsthat bind to the recombinant viral vector. In one embodiment, ADAs that bind to the recombinant viral vector are absent from the individual prior to and/or after administration of the TKI. Methods to measure ADAs prior to and/or after administration of a recombinant viral vector are known in the art and also herein described.

In one embodiment, the AD As that bind to the recombinant viral vector are reduced by more than 20%, 30%, 40%, 50%, 60%, 70%, 80%, 90%, 100%, optionally as compared to natural history data of a relevant control group without administration of a tyrosine kinase inhibitor (TKI). In one embodiment the reduction of ADAs is measured in the serum of an individual. The reduction of the serum level of ADAsis in particular as compared to the serum level in an individual (including the same individual) without administration of the TKI (i.e. in such case the serum level is reduced as compared to the serum level without/before administration of the TKI). Said reduction of the serum level is in particular as compared to the serum level or cytokine secretion in an individual (including the same individual) with administration (in particular first administration) of the recombinant viral vector but without administration of the TKI (i.e. in such case the serum level is reduced as compared to the serum level with/after administration of the recombinant viral vector but without/before administration of the TKI). Without said reduction, the serum level (of ADAs) may be elevated/increased in relation to the (administration of) the recombinant viral vector. In some aspects, said reduction is clinically meaningful and/or statistically significant. Said reduction may be partial or complete. In some aspects, said reduction is clinically meaningful and/or statistically significant.

In one embodiment, the individual is at risk of developing ADAs to the polypeptide encoded by said heterologous polynucleotide. ADAs to the heterologous polypeptide can decrease the therapeutic efficacy, for example by lowering the number of cells expressing the heterologous polypeptide (i.e. the transgene).

In one embodiment, transgene expression is increased upon re-administration of the viral vector, in particular as compared to the transgene expression prior to re-administration of the viral vector. In one embodiment, transgene expression is increased by more than 10%, 20%, 30%, 40%, 50%, 60%, 70%, 80%, 90%, or 100% as compared to the transgene expression prior to re-administration of the viral vector. In one embodiment, the transgene expression is maintained upon re-administration of the viral vector, in particular as compared to the transgene expression prior to re-administration of the viral vector. In one embodiment the increase of transgene expression is measured in the serum and/or in the (target) tissue of an individual. The increase of transgene expression is in particular as compared to the transgene expression in an individual (including the same individual) without administration of the TKI (i.e. in such case the transgene expression is reduced as compared to the transgene expression without/before administration of the TKI). Said increase of transgene expression is in particular as compared to the transgene expression in an individual (including the same individual) with administration (in particular first administration) of the recombinant viral vector but without administration of the TKI (i.e. in such case the transgene expression is increased as compared to the transgene expression with/after administration of the recombinant viral vector but without/before administration of the TKI). Without said increase, the transgene expression may be lowered/decreased in relation to the (administration of) the recombinant viral vector. In some aspects, said increase is clinically meaningful and/or statistically significant. Said increase may be partial or complete. In some aspects, said increase is clinically meaningful and/or statistically significant.

Sequences

| Name | Sequence | [symbol] |
|---|---|---|
| hSEAP | MVLGPCMLLLLLLLGLRLQLSLGIIPVEEENPDFWNREAAEALGAAKKLQPAQTAAK | 1 |
| | NLIIFLGDGMGVSTVTAARILKGQKKDKLGPEIPLAMDRFPYVALSKTYNVDKHVPD | |
| | SGATATAYLCGVKGNFQTIGLSAAARFNQCNTTRGNEVISVMNRAKKAGKSVGVVTT | |
| | TRVQHASPAGTYAHTVNRNWYSDADVPASARQEGCQDIATQLISNMDIDVILGGGRK | |
| | YMFRMGTPDPEYPDDYSQGGTRLDGKNLVQEWLAKRQGARYVWNRTELMQASLDPSV | |
| | THLMGLFEPGDMKYEIHRDSTLDPSLMEMTEAALRLLSRNPRGFFLFVEGGRIDHGH | |
| | HESRAYRALTETIMEDDAIERAGQLTSEEDTLSLVTADHSHVFSFGGYPLRGSSIFG | |
| | LAPGKARDRKAYTVLLYGNGPGYVLKDGARPDVTESESGSPEYRQQSAVPLDEETHA | |
| | GEDVAVFARGPQAHLVHGVQEQTFIAHVMAFAACLEPYTACDLAPPAGTTDAAHPGR | |
| | SRSKRLD | |
| AAV8VP1 | MAADGYLPDWLEDNLSEGIREWWALKPGAPKPKANQQKQDDGRGLVLPGYKYLGPFN | 2 |
| | GLDKGEPVNAADAAALEHDKAYDQQLQAGDNPYLRYNHADAEFQERLQEDTSFGGNL | |
| | GRAVFQAKKRVLEPLGLVEEGAKTAPGKKRPVEPSPQRSPDSSTGIGKKGQQPARKR | |
| | LNFGQTGDSESVPDPQPLGEPPAAPSGVGPNTMAAGGGAPMADNNEGADGVGSSSGN | |
| | WHCDSTWLGDRVITTSTRTWALPTYNNHLYKQISNGTSGGATNDNTYFGYSTPWGYF | |
| | DFNRFHCHFSPRDWQRLINNNWGFRPKRLSFKLFNIQVKEVTQNEGTKTIANNLTST | |
| | IQVFTDSEYQLPYVLGSAHQGCLPPFPADVFMIPQYGYLTLNNGSQAVGRSSFYCLE | |
| | YFPSQMLRTGNNFQFTYTFEDVPFHSSYAHSQSLDRLMNPLIDQYLYYLSRTQTTGG | |
| | TANTQTLGFSQGGPNTMANQAKNWLPGPCYRQQRVSTTTGQNNNSNFAWTAGTKYHL | |
| | NGRNSLANPGIAMATHKDDEERFFPSNGILIFGKQNAARDNADYSDVMLTSEEEIKT | |
| | TNPVATEEYGIVADNLQQQNTAPQIGTVNSQGALPGMVWQNRDVYLQGPIWAKIPHT | |
| | DGNFHPSPLMGGFGLKHPPPQILIKNTPVPADPPTTFNQSKLNSFITQYSTGQVSVE | |
| | IEWELQKENSKRWNPEIQYTSNYYKSTSVDFAVNTEGVYSEPRPIGTRYLTRNL | |
| hFIX | MQRVNMIMAESPGLITICLLGYLLSAECTVFLDHENANKILNRPKRYNSGKLEEFVQ | 3 |
| | GNLERECMEEKCSFEEAREVFENTERTTEFWKQYVDGDQCESNPCLNGGSCKDDINS | |
| | YECWCPFGFEGKNCELDVTCNIKNGRCEQFCKNSADNKVVCSCTEGYRLAENQKSCE | |
| | PAVPFPCGRVSVSQTSKLTRAETVFPDVDYVNSTEAETILDNITQSTQSFNDFTRVV | |
| | GGEDAKPGQFPWQVVLNGKVDAFCGGSIVNEKWIVTAAHCVETGVKITVVAGEHNIE | |
| | ETEHTEQKRNVIRIIPHHNYNAAINKYNHDIALLELDEPLVLNSYVTPICIADKEYT | |
| | NIFLKFGSGYVSGWGRVFHKGRSALVLQYLRVPLVDRATCLRSTKFTIYNNMFCAGF | |
| | HEGGRDSCQGDSGGPHVTEVEGTSFLTGIISWGEECAMKGKYGIYTKVSRYVNWIKE | |
| | KTKLT | |
| AAV2VP1 | MAADGYLPDWLEDTLSEGIRQWWKLKPGPPPPKPAERHKDDSRGLVLPGYKYLGPFN | 4 |
| | GLDKGEPVNEADAAALEHDKAYDRQLDSGDNPYLKYNHADAEFQERLKEDTSFGGNL | |
| | GRAVFQAKKRVLEPLGLVEEPVKTAPGKKRPVEHSPVEPDSSSGTGKAGQQPARKRL | |
| | NFGQTGDADSVPDPQPLGQPPAAPSGLGTNTMATGSGAPMADNNEGADGVGNSSGNW | |
| | HCDSTWMGDRVITTSTRTWALPTYNNHLYKQISSQSGASNDNHYFGYSTPWGYFDFN | |
| | RFHCHFSPRDWQRLINNNWGFRPKRLNFKLFNIQVKEVTQNDGTTTIANNLTSTVQV | |
| | FTDSEYQLPYVLGSAHQGCLPPFPADVFMVPQYGYLTLNNGSQAVGRSSFYCLEYFP | |
| | SQMLRTGNNFTFSYTFEDVPFHSSYAHSQSLDRLMNPLIDQYLYYLSRTNTPSGTTT | |
| | QSRLQFSQAGASDIRDQSRNWLPGPCYRQQRVSKTSADNNNSEYSWTGATKYHLNGR | |
| | DSLVNPGPAMASHKDDEEKFFPQSGVLIFGKQGSEKTNVDIEKVMITDEEEIRTTNP | |
| | VATEQYGSVSTNLQRGNRQAATADVNTQGVLPGMVWQDRDVYLQGPIWAKIPHTDGH | |
| | FHPSPLMGGFGLKHPPPQILIKNTPVPANPSTTFSAAKFASFITQYSTGQVSVEIEW | |
| | ELQKENSKRWNPEIQYTSNYNKSVNVDFTVDINGVYSEPRPIGTRYLTRNL | |
| AAV9VP1 | MAADGYLPDWLEDNLSEGIREWWALKPGAPQPKANQQHQDNARGLVLPGYKYLGPGN | 5 |
| | GLDKGEPVNAADAAALEHDKAYDQQLKAGDNPYLKYNHADAEFQERLKEDTSFGGNL | |
| | GRAVFQAKKRLLEPLGLVEEAAKTAPGKKRPVEQSPQEPDSSAGIGKSGAQPAKKRL | |
| | NFGQTGDTESVPDPQPIGEPPAAPSGVGSLTMASGGGAPVADNNEGADGVGSSSGNW | |
| | HCDSQWLGDRVITTSTRTWALPTYNNHLYKQISNSTSGGSSNDNAYFGYSTPWGYFD | |
| | FNRFHCHFSPRDWQRLINNNWGFRPKRLNFKLFNIQVKEVTDNNGVKTIANNLTSTV | |
| | QVFTDSDYQLPYVLGSAHEGCLPPFPADVFMIPQYGYLTLNDGSQAVGRSSFYCLEY | |
| | FPSQMLRTGNNFQFSYEFENVPFHSSYAHSQSLDRLMNPLIDQYLYYLSKTINGSGQ | |
| | NQQTLKFSVAGPSNMAVQGRNYIPGPSYRQQRVSTTVTQNNNSEFAWPGASSWALNG | |
| | RNSLMNPGPAMASHKEGEDRFFPLSGSLIFGKQGTGRDNVDADKVMITNEEEIKTTN | |
| | PVATESYGQVATNHQSAQAQAQTGWVQNQGILPGMVWQDRDVYLQGPIWAKIPHTDG | |
| | NFHPSPLMGGFGMKHPPPQILIKNTPVPADPPTAFNKDKLNSFITQYSTGQVSVEIE | |
| | WELQKENSKRWNPEIQYTSNYYKSNNVEFAVNTEGVYSEPRPIGTRYLTRNL | |

[symbol]

indicates data missing or illegible when filed

### BRIEF DESCRIPTION OF THE DRAWINGS

FIG. 1. rAAV8 readministration in mice and Dasatinib treatment. C57B1/6 mice were intravenously dosed on study day 1 with a rAAV8 encoding the hSEAP (human secreted alkaline phosphatase) at the dose of 1E12 vg/kg. On study day 22, they received a second rAAV8 encoding hFIX (human Factor IX) at the dose of 5E13 vg/kg. Group 1 (non-immunized mice, n=9) did not receive the first rAAV8 injection. Group 2 (immunized mice, n=10) received both rAAV8 injections. Group 3 mice (n=10) received the two rAAV8 injections and were treated by oral gavage with 50 mg/kg dasatinib twice per day for 7 days starting on the day of first rAAV8 dosing. Blood samples were collected one day before and every 7 days after the first rAAV8 treatment.

FIGS. 2A-2C. Effects of dasatinib treatment on anti-AAV8 IgM titers. Serum was isolated from blood collected on study days 0, 8, 15, 21, 29 and 36 (FIG. 1) and anti-AA V8 IgM measured by ELISA, performed on immobilized empty AAV8 capsids. IgM antibody titers were determined by ELISA OD values of serial dilutions (1:10 and 7-step serial dilution 1:3). For the determination of mean background level, readouts were pooled from all mouse groups at day 0. The positivity threshold is represented by the dotted line. The kinetics of anti-AAV8 IgM antibody titers is shown for each mouse group (FIGS. 2A-2C). The empty circles represent titers under the threshold, and crossed circles represent titers above the threshold.

FIGS. 3A-3C. Effects of dasatinib treatment on anti-AAV8 IgG titers. Serum was isolated from blood collected on study days 0, 8, 15, 21, 29 and 36 (FIG. 1) and anti-AAV8 IgG measured by ELISA, performed on immobilized empty AAV8 capsids. IgG antibody titers were determined by ELISA OD values of serial dilutions (1:10 and 7-step serial dilution 1:3). For the determination of mean background level, readouts were pooled from all mouse groups at day 0. The positivity threshold is represented by the dotted line. The kinetics of anti-AA V8 IgG antibody titers is shown for each mouse group (FIGS. 3A-3C). The empty circles represent titers under the threshold, and crossed circles represent titers above the threshold.

FIGS. 4A-4B. Dasatinib treatment allows transgene expression following rAAV re-administration. Serum was isolated from blood collected on study days 0, 8, 15, 21, 29 and 36 (FIG. 1). (FIG. 4A) Expression of the first transgene hSEAP was measured by chemiluminescence. (FIG. 4B) After AAV8 re-administration, expression of the second transgene hFactorIX was measured by ELISA. Mean concentrations are shown +/−SD.

FIGS. 5A-5B. Effect of dasatinib on AAV-mediated cytokine release in human whole blood. The production of low levels of IFN-γ and IL-6 in whole blood are stimulated after 24 hrs incubation with 5E11 vg/mL of AAV8-hSEAP. In the presence of dasatinib at 12.5 or 50 nM, this production was reduced dose-dependently. Depicted are cytokine concentrations (pg/mL+/−SD, left), fold increases over the level measured in the PBS control (middle) and percentages of inhibition of AAV-dependent cytokine release (right, calculated as 100 × (concentration with AAV8 alone-concentration with AAV8 and dasatinib)/(concentration with AAV8 alone)), respectively. (FIG. 5A) Interferon-γ production. (FIG. 5B) IL-6 production.

FIGS. 6A-6C: Dasatinib inhibits AA V8-dependent cytokine release in human whole blood. Fresh whole blood from 4 healthy donors was incubated with either PBS or AAV8-hSEAP (5e11 vg/mL) with IVIG (PRIVIGEN, 1/100) for 24 hrs. Dasatinib was added at the concentration of 50 nM 1 hr before addition of AAV8. In some of the culture wells, a second dose of dasatinib was added 9 hrs after AAV8 treatment (dasa 50 nM 2x). After 24 hrs, the plasma was collected and cytokines measured using Quanterix kits. Depicted are the cytokine fold increases over PBS for IFN-γ and TNF-α (FIG. 6A), IL-6 and IL-1x (FIG. 6B), and IL-2 and IL-1β (FIG. 6C), with the percentages of inhibition in the presence of dasatinib (means +/−SEM).

FIG. 7: In vivo study to assess the impact of prolonging the dasatinib treatment. All mouse groups, except Group 1, were immunized on day 1 with an i.v. injection of AAV8-hSeap (1e12 vg/kg). Four groups received an i.v. injection of AAV8-FactorIX (3e13 vg/kg) on day 43. Concomitantly to the first AAV administration, Groups 3 and 4 were treated with dasatinib for either 1 or 2 weeks, respectively. Dasatinib was administered at the dose of 50 mg/kg per oral gavage, twice per day and starting 1 hr before the first AAV treatment.

FIGS. 8A-8B: Prolongation of dasatinib treatment from 1 week to 2 weeks improves the inhibition of IgM formation to AAV8. In the experiment described in FIG. 8A and FIG. 8B anti-AAV8 IgM titers (median+/−SD) were measured in each mouse group from day 0 (before AAV8-hSeap administration) to day 64, as described for FIGS. 2A-2C. Each circle represents an individual IgM titer.

FIGS. 9A-9B: Prolongation of dasatinib treatment from 1 week to 2 weeks improves the inhibition of IgG formation to AAV8. In the experiment described in FIG. 9A and FIG. 9B, anti-AAV8 IgG titers (median+/−SD) were measured in each mouse group from day 0 (before AAV8-hSeap administration) to day 64, as described for FIGS. 3A-3C. Each circle represents an individual IgG titer.

FIG. 10: Prolongation of dasatinib treatment upon first AAV8 dosing improves transgene expression after readministration. In the experiment described in FIG. 10, the levels of hFIX expression (ng/mL, median+/−SD) were measured for each group, starting on day 42 (one day before administration of AAV8-hFIX) and up to day 64.

FIG. 11: Dasatinib treatment inhibits cytokine and chemokine release by murine splenocytes in response of AAV8. Splenocytes were isolated from a C57/B16 mouse and incubated for 24 hrs with AAV8-hSeap at MOI 1E5 in the presence or absence of dasatinib (100 nM, 50 nM or 12.5 nM). Cytokines and chemokines were measured in the culture supernatants. LPS (positive) and PBS (negative) controls are shown on the graphs. Means+/−SD (triplicates). Dashed line: Lower limit of quantification.

FIGS. 12A-12B: Dasatinib treatment reduces T cell responses to AAV2 and AAV9 in vitro. PBMCs from human heathy donors were stimulated with peptide pools covering the AAV2 and AAV9 capsid sequences in the absence (black bars) or presence of 100 nM dasatinib (grey bars). After 48 hrs, IFN-γ- and TNF-α secreting cells were measured by Fluorospot. Mean Spot-Forming Cellsmedian+/−SD are shown for 1E6 PBMCs. Dotted line: Positivity threshold.

### EXAMPLES

The following are examples of methods and compositions of the invention. It is understood that various other aspects may be practiced, given the general description provided above.

### Example 1. Dasatinib Prevents Anti-AAV8 Antibody Formation After rAAV8 Administration in Mice

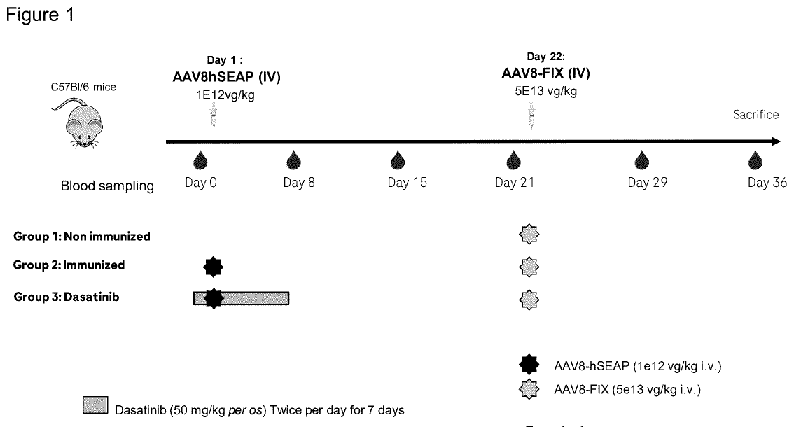

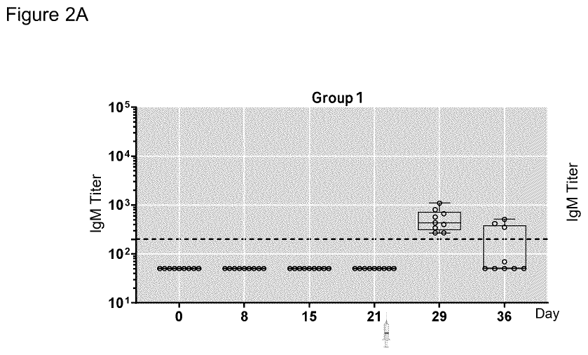

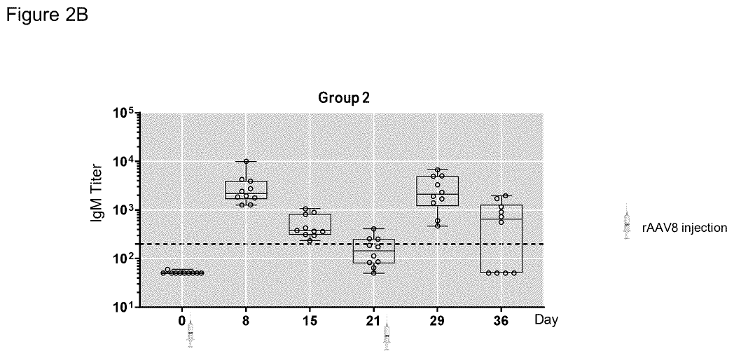

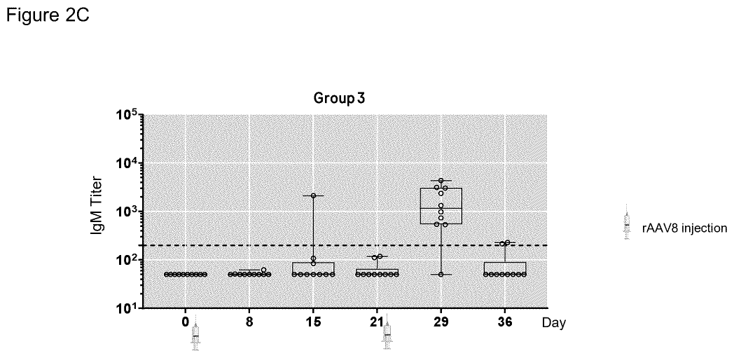

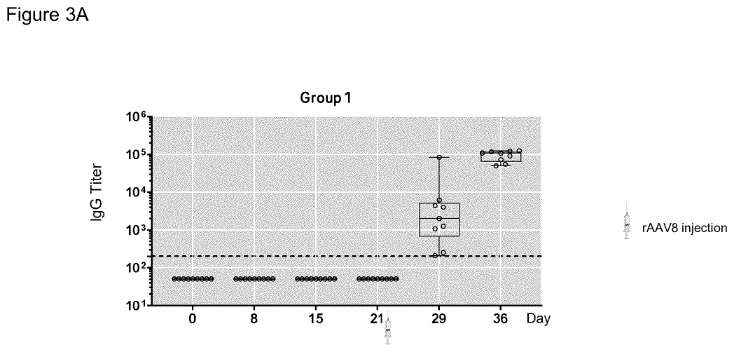

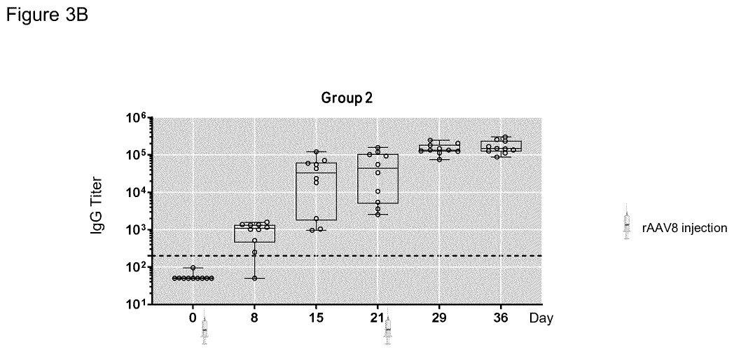

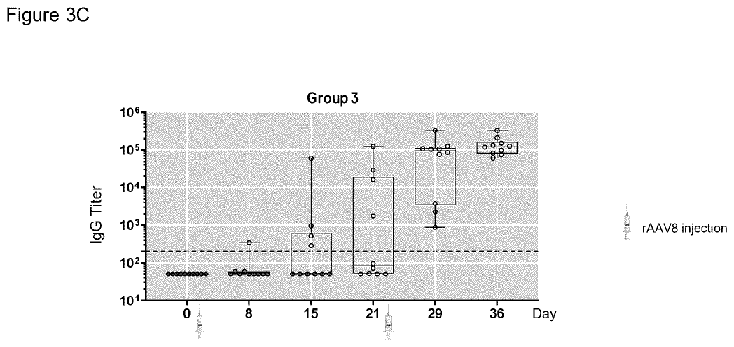

To assess whether dasatinib could efficiently prevent anti-AAV antibody formation after rAAV administration and allow efficient re-administration of rAAV, a study was conducted in mice (FIG. 1). Groups of C57B1/6 mice were intravenously dosed with a first rAAV8 encoding hSEAP (human secreted alkaline phosphatase, under control of a CMV promoter) at the dose of 1E12vg/kg on study day 1. On study day 22, they were dosed with a second rAAV8 encoding hFIX (human Factor IX, under control of a CMV promoter) at the dose of 5E13 vg/kg. The non-immunized control Group 1 did not receive the first rAAV8 injection, while the immunized control Group 2 received the two injections. Group 3 mice were treated with Dasatinib at the dose of 50 mg/kg by oral gavage twice per day for 7 days, starting on the day of first rAAV8 dosing. Blood samples were collected on study days 0 (one day before first AAV dosing), 8, 15, 21, 29 and 36. Serum IgM and IgG directed against the AAV8 capsid were titrated in the serum samples (FIGS. 2A-2C and 3A-3C). The peaks of anti-AAV8 IgM were observed one week after first rAAV8 dosing, on day 8 for the immunized control Group 2 and on day 29 for the non-immunized control Group 1 (10/10 mice above the threshold) (FIGS. 2A-2C). In the dasatinib-treated Group 3, serum from all mice remained underthe IgM positivity threshold until administration of the second rAAV8. IgM positivity of mouse 302 only on day 15 was attributed to a technical issue in ELISA. Anti-AA V8 IgG titers were drastically reduced in the dasatinib-treated Group 3 compared with the immunized control Group 2. On study day 21, only 4/10 Group 3 mice were above the positivity threshold while 10/10 Group 2 mice were above this threshold (FIGS. 3A-3C). On day 36, two weeks after rAAV8 re-administration, the anti-AAV8 IgG titers were comparable in all mouse groups, showing that the effect of dasatinib was transient and did not affect a subsequent immune response to rAAV8. These data show that dasatinib efficiently reduces anti-AAV8 antibody formation after rAAV8 administration in mice.

### Example 2. Transient Dasatinib Treatment Allows Transgene Expression Following rAAV8 re-Administration

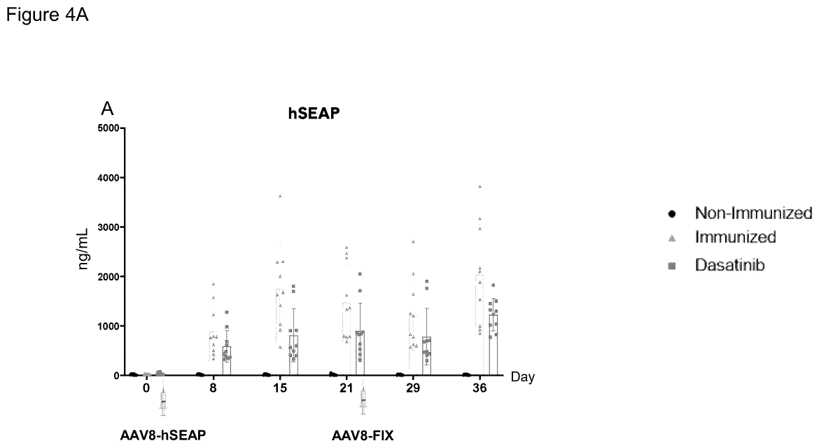

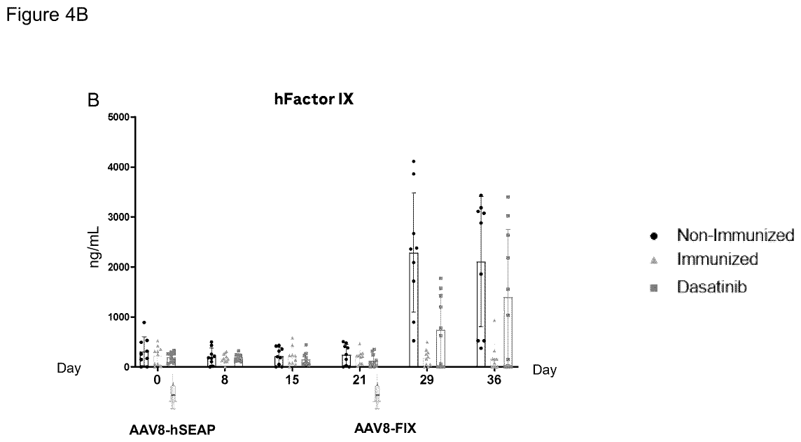

Expression of the first transgene hSEAP and second transgene hFIX were measured in the serum samples from the experiment presented above (FIG. 1, Example 1). As expected, human SEAP was detected in the serum of mice from Groups 2 and 3, already 7 days after injection of AAV8-hSEAP on study day 1 (FIG. 4A). Following second administration of rAAV8 on study day 22, hFIX transgene expression was not detectable in the serum of mice from the immunized control Group 2 (FIG. 4B) and remained at baseline until day 36 (14 days after AAV8-hFIX dosing), showing that re-administration had been totally inefficient. In contrast, hFIX expression was detected in the serum of all mice from the non-immunized Group 1 (mean: 2107 ng/ml) as well as in most mice from the dasatinib-treated Group 3 (average: 1398 ng/ml). This data shows that dasatinib treatment at the time of first rAAV dosing restored at least partially the efficacy of transduction by a second rAAV. Altogether, results from Examples 1 and 2 show that the inhibition of anti-AA V8 antibody formation resulting from transient dasatinib treatment allows for efficient AAV re-administration.

### Example 3. Dasatinib Dose-Dependently Inhibits AAV-Induced Cytokine Production in Human Blood

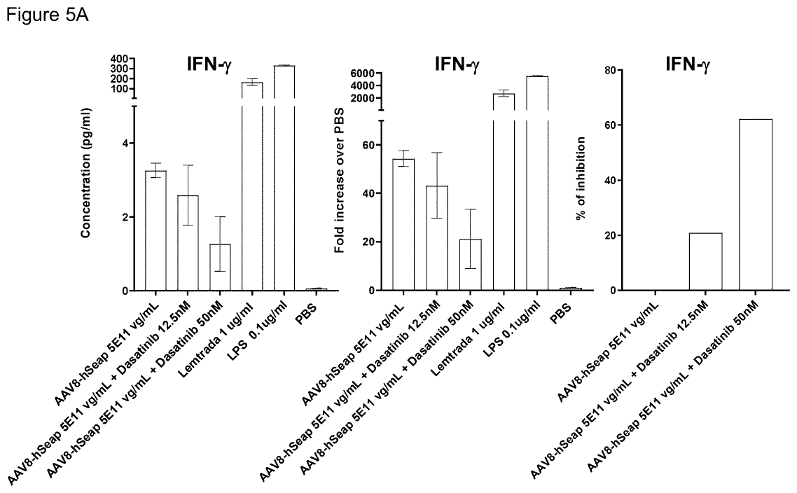

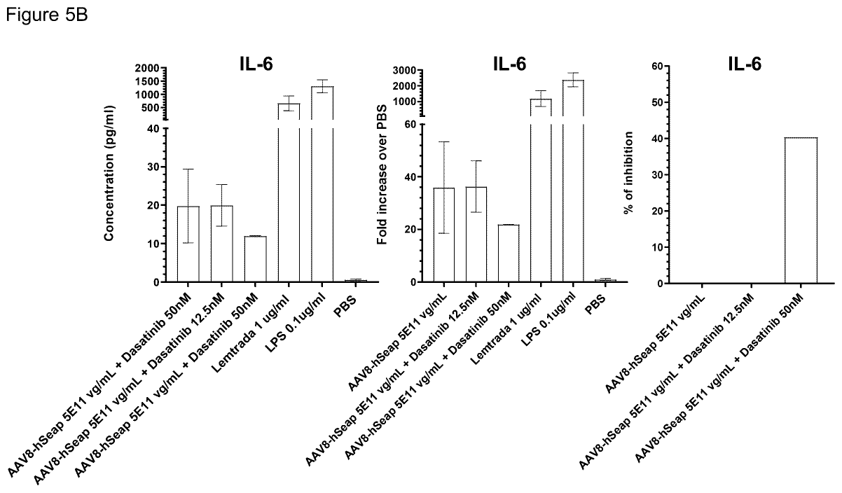

To assess whether dasatinib could affect AAV-mediated cytokine release, an assay was performed in whole blood from a healthy donor. This donor was seropositive for anti-AAV8 antibodies (IgG titer: 1/21870; IgM titer: 1/810). Whole blood from an AAV8 pre-immune donor was incubated in triplicates with 5E11 vg/mL (GC/mL) of an AAV8-hSEAP vector, in the presence or absence of 12.5 nM or 50 nM dasatinib. Lipopolysaccharide (LPS) and Lemtrada (alemtuzumab, anti-CD52, Genzyme), a monoclonal antibody known to induce strong cytokine release in blood, were used as positive controls at a concentration of 1 μg/ml and 0.1 μg/ml respectively. PBS was used as a negative control and to assess baseline cytokine production. Dasatinib was added to the blood 1 hr before incubation with AAV8-hSEAP. After 24 hrs, plasma supernatants were collected and cytokines measured using Quanterix kits with the SP-X imaging and analysis system (Simoa). FIGS. 5A and 5B show that the production of low levels of IFN-γ and IL-6 in whole blood are stimulated after 24 hrs incubation with 5E11 vg/mL of AAV8-hSEAP. In the presence of dasatinib at 12.5 or 50 nM, this production was reduced dose-dependently. These results suggest that inhibition of cytokine release could be a mechanism by which dasatinib inhibits anti-AAV antibody production.

### Example 4. Dasatinib Dose-Dependently Inhibits AAV-Induced Cytokine Production in Human Blood

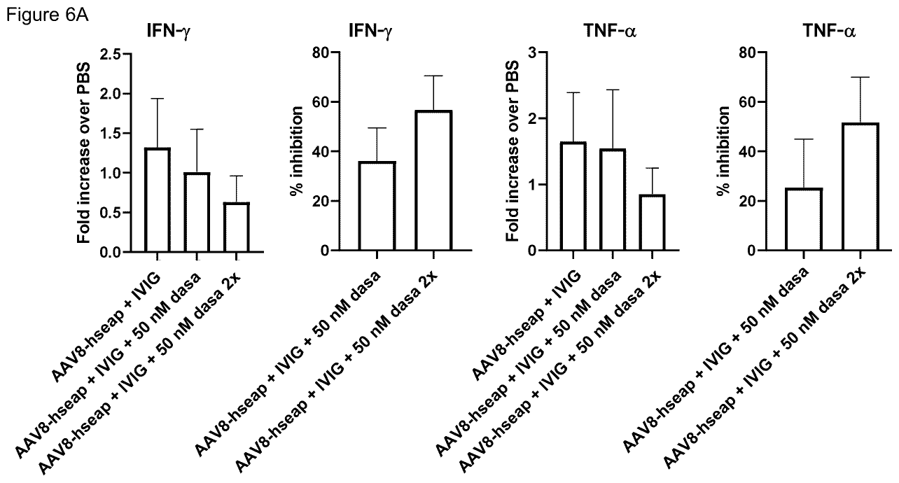

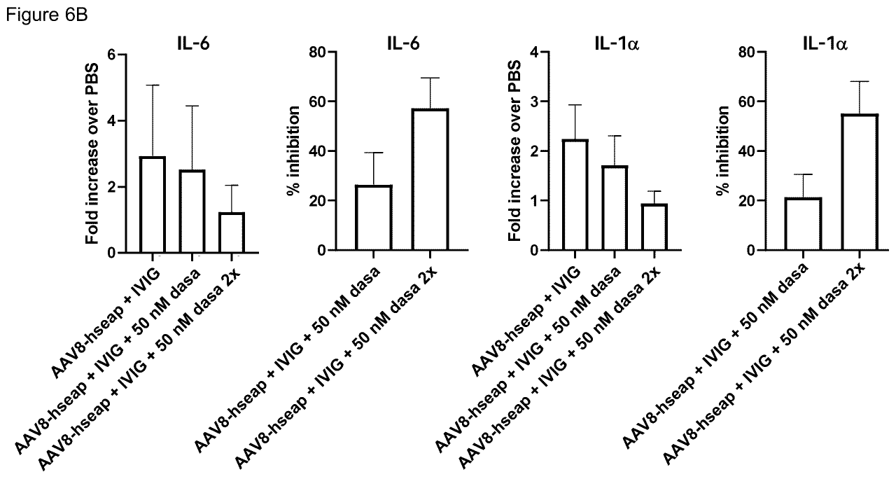

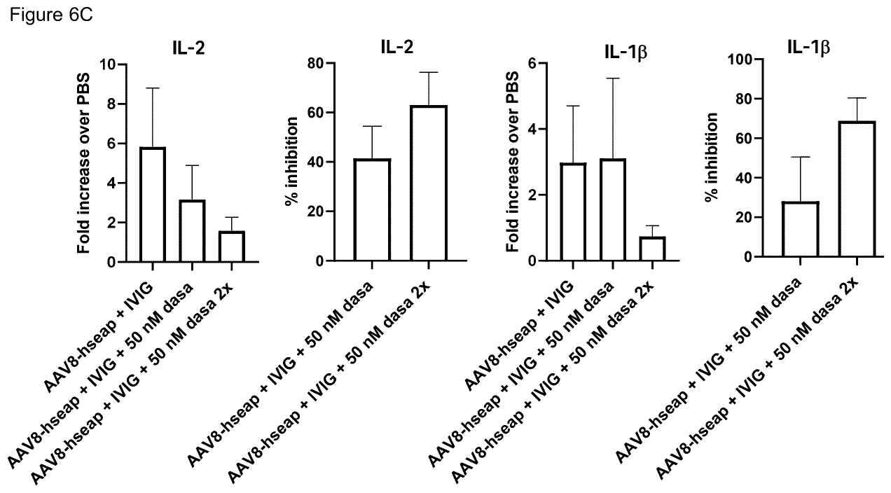

To study in more detail the effects of dasatinib on AAV-dependent cytokine release, another whole blood assay was performed. Whole blood from four healthy donors was incubated with AAV8-hSEAP (5e11 vg/ml) for 24 hrs. Intravenous Immunoglobulin (IVIG, Privigen) which contains anti-AAV antibodies was added to the blood samples. Dasatinib was added at 50 nM 1 hr before the AAV treatment, PBS was used as a negative control. As the half-life of dasatinib is very short (Lindauer M et al, Recent Results Cancer Res. 2010;184:83-102), a second dasatinib addition was performed 9 hrs after the AAV treatment. After 24 hrs, plasma supernatants were collected and cytokines measured using Quanterix kits. FIGS. 6A-6C show that the production of IFN-Y, IL-6, IL-2, TNF-α, IL-la and IL-1β was inhibited in the presence of dasatinib. This inhibition was more pronounced when dasatinib was added both 1 hr before and 9 hrs after AAV treatment. These results show that dasatinib inhibits the release of several pro-inflammatory cytokines including IL-6 and IL-1B, which have been reported to stimulate the antibody response to AAV capsid (Kuranda K et al, J Clin Invest. 2018 128(12):5267-5279). Inhibition of AAV-mediated cytokine release likely contributes to the effect of dasatinib on antibody formation.

### Example 5. Prolongation of Dasatinib Treatment Improves Anti-AAV8 Antibody Inhibition After rAAV8 Administration and Allows Efficient Re-Dosing in Mice

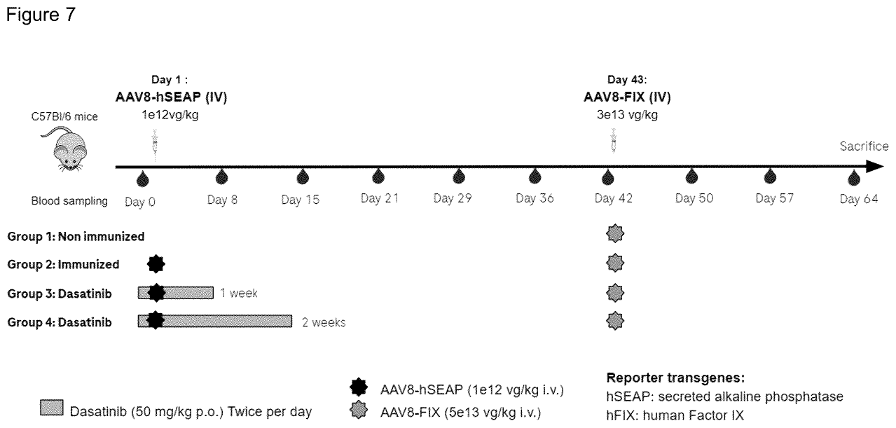

In the experiment described in Example 1, despite the strong inhibition of anti-AAV antibody formation observed in most of the mice, 4 out of 10 dasatinib-treated animals started to develop anti-AAV8 IgG before re-dosing (FIG. 3C, days 15-21). The presence of residual AAV8 particles in the circulation and in the tissues after the cessation of the dasatinib treatment might lead to this antibody formation. For this reason, we investigated whether prolonging the dasatinib treatment might more efficiently inhibit the humoral response to the AAV8 capsid. To this purpose, the same protocol was applied as in Example 1, but using either a 1-week or a 2-week dasatinib treatment. In addition, re-dosing was performed at a later time point (day 43 instead of day 22) to be able to observe potential antibody formation after the cessation of dasatinib treatment. This protocol is depicted in FIG. 7.

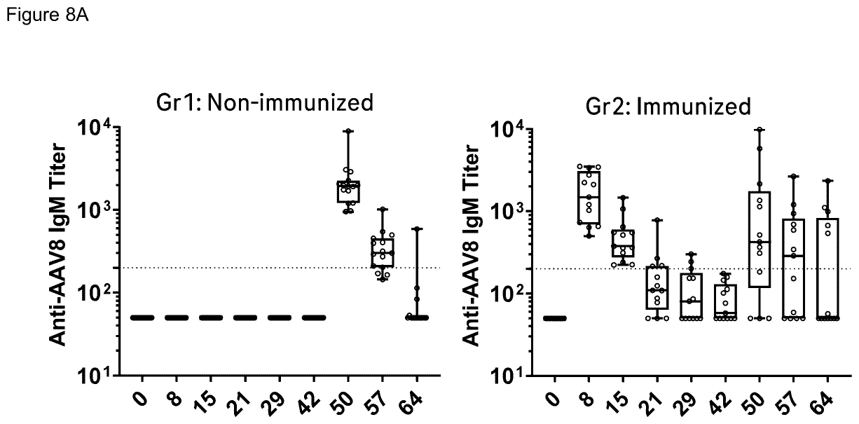

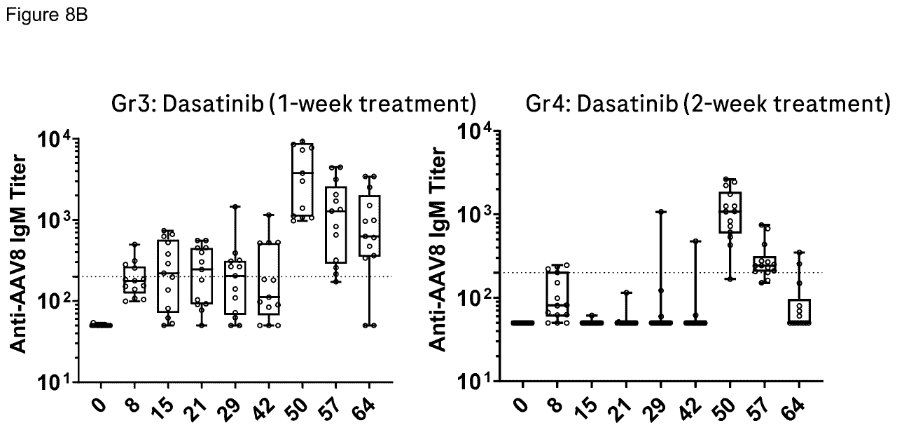

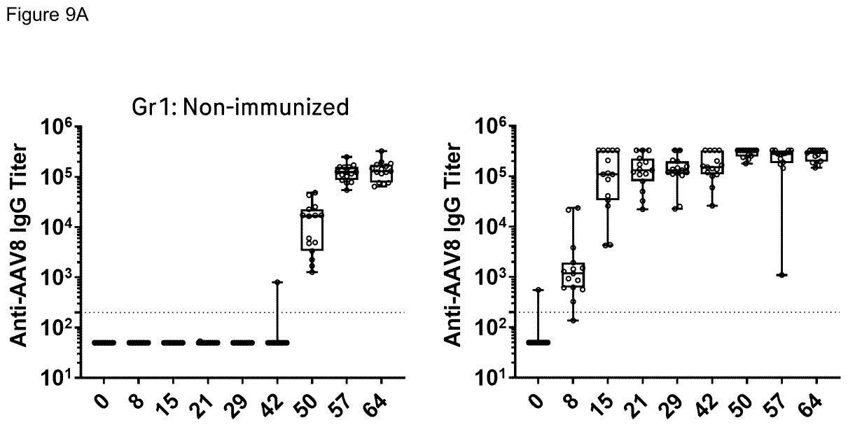

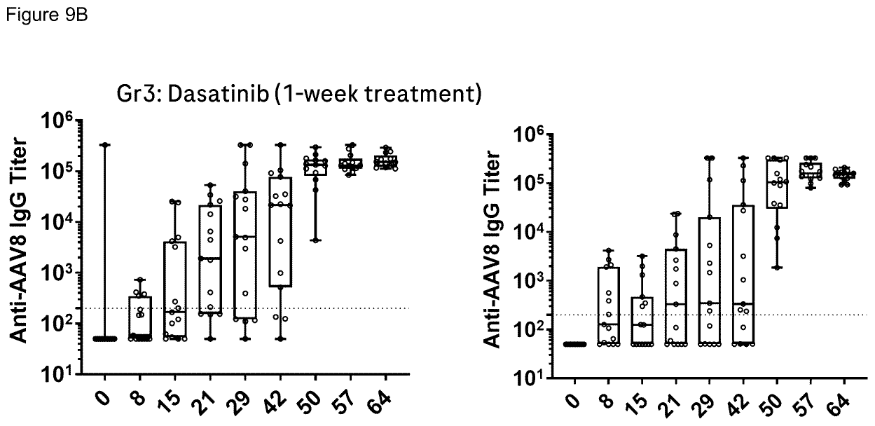

Circulating IgM and IgG directed against the AAV8 capsid were titrated in the serum samples (FIGS. 8A-8B and 9A-9B). The peaks of anti-AAV8 IgM were observed one week after first rAAV8 dosing, on day 8 for the immunized control Group 2 and on day 50 for the non-immunized control Group 1 (10/10 mice above the threshold) (FIGS. 8A-8B). In Group 3 treated with dasatinib for 1 week, IgM titers remained low to negative until administration of the second rAAV8. Four out of 15 mice had positive IgM titers to AAV8 on day 42, before AAV8re-dosing. Inhibition of IgM production was stronger in Group 4, treated with dasatinib for 2 weeks. In this group, only one mouse showed a positive IgM titer on day 42.

In Group 3 treated with dasatinib for 1 week, 3/15 mice showed negative anti-AAV8 IgG titers on day 42, while all 15 mice of the immunized control Group 2 had very high IgG titers (FIGS. 9A-9B). As for IgM, inhibition of IgG formation was stronger in Group 4 mice treated with dasatinib for 2 weeks. In this group, 5/15 mice had IgG titers below the positivity threshold on day 42 and the median titer was much lower than in Group 3. In conclusion, for both IgM and IgG formation to AAV8, prolongation of the dasatinib treatment from 1 to 2 weeks resulted in more potent inhibition.

On day 64, 3 weeks after rAAV8 re-administration, the anti-AAV8 IgG titers were comparable in all mouse groups, again showing that the effect of dasatinib was transient and did not affect a subsequent immune response to rAAV8.

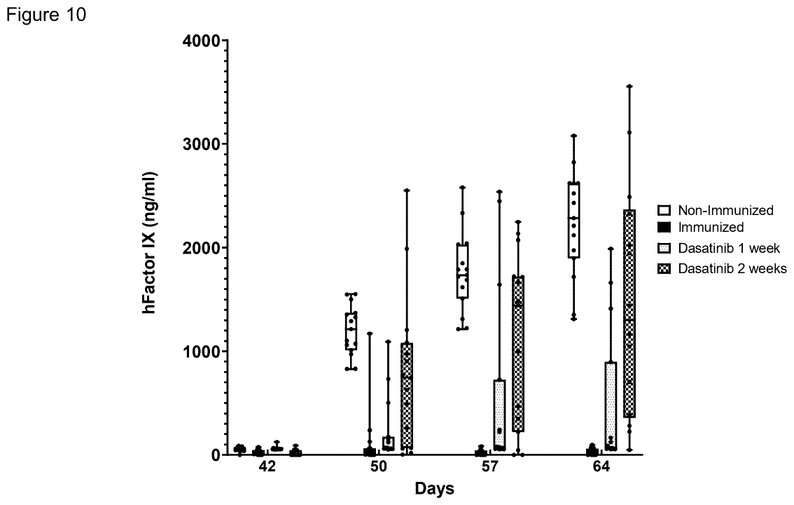

The inhibition of antibody formation to AAV8 correlated with the improvement of hFIX transgene expression after re-dosing, showing efficient transduction by the second AAV8(FIG. 10). Expression of hFIX was inversely correlated with the IgG and IgM titers measured on day 42 before re-dosing. In the mice that had remained IgG-negative on day 42, hFIX levels on day 64 were in the range of those measured in the non-immunized control Group 1.

Expression of hFIX was higher in Group 4 treated with dasatinib for 2 weeks, compared Group 3 treated for 1 week only, reflecting the more complete inhibition of anti-AAV8 antibody formation.

### Example 6: Dasatinib Reduces AAV8-Dependent Cytokine and Chemokine Release in Murine Splenocytes

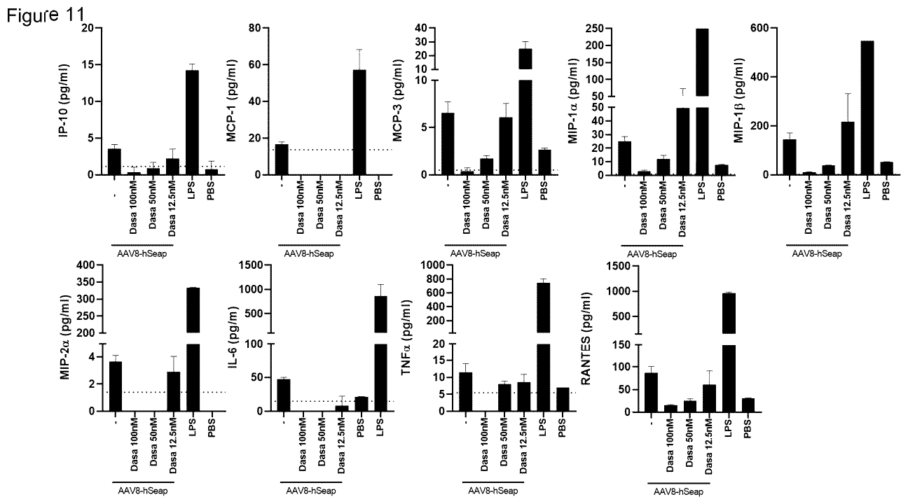

To investigate the mechanism of dasatinib-mediated inhibition of the humoral response to AAV8 observed in mice (Examples 1 and 5), we looked whether the rapid production of cytokines and chemokines by immune cells was affected by the dasatinib treatment. Total splenocytes from a C57BL/6 mouse were cultured for 24 hrs in the presence or absence of dasatinib at different concentrations. Cytokines and chemokines secreted in the culture supernatants were analyzed using ProcartaPlex Immunoassays (Luminex). As shown on FIG. 11, dose-dependent decreases were measured for a range of cytokines (IL-6, TNF-α) and chemokines (IP-10/CXCL10, MCP-1, MCP-3, MIP-1α, MIP-1β, MIP-2α) characteristic of the early innate immune response to rAAV (Shirley J L et al. Mol Ther. 2020 Mar. 4;28 (3): 709-722).

Together with Examples 3 and 4 exemplifying the effects of dasatinib on cytokine production by human PBMCs, these results show that dasatinib down-modulates innate immune responses to AAV both in human and mice. They also suggest that the inhibition of antibody response to AAV vectors observed in mice may be, at least in part, a consequence of this inhibition of early cytokine and chemokine production. In particular, IL-6 release has been shown to contribute to anti-AAV antibody formation (Kuranda K et al, J Clin Invest. 2018 Dec. 3;128(12):5267-5279).

### Example 7. Dasatinib Inhibits Human T Cell Responses to AAV2 and AAV9 in Vitro

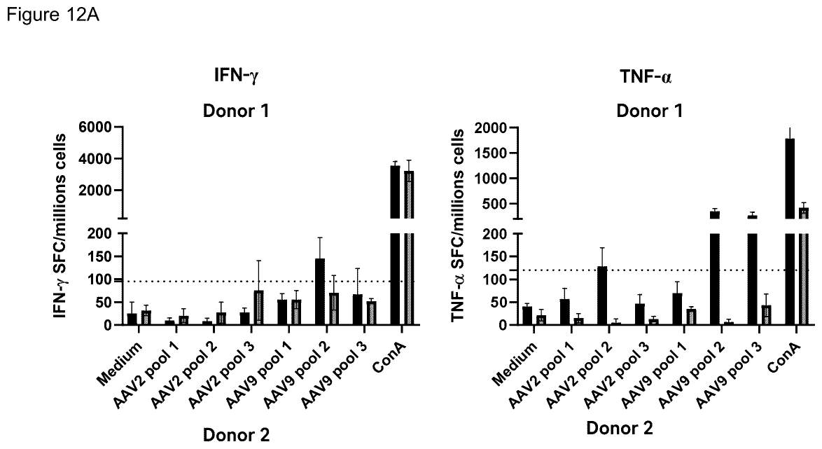

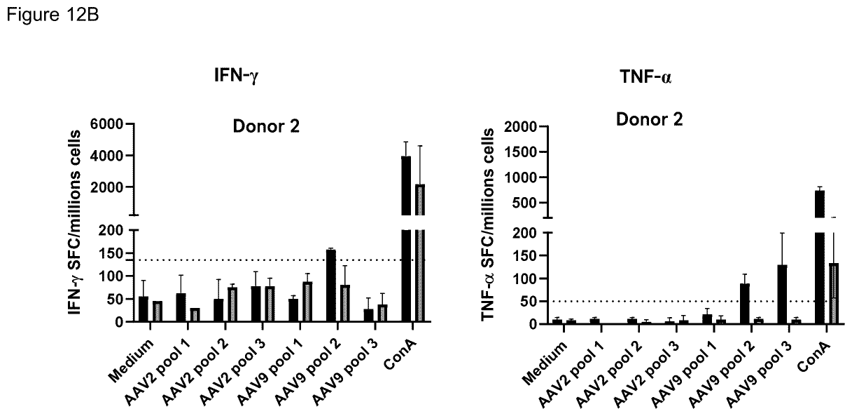

The above Examples show that dasatinib treatment has the potential to inhibit both innate and humoral immune responses to AAV8. An important question was its potential effect on the cellular adaptive immune response. T cell responses to rAAV have been reported to induce the clearance of AAV-transduced cells and thereby decrease the duration of transgene expression. It is also known that T cell responses to AAV can mediate liver toxicities in the clinic. T cell responses to AAV can be measured in healthy human blood donors who have previously been infected with wild-type AAV. FluoroSpot assays reveal the proportions of IFN-y-and TNF-a-producing PBMCs induced upon incubation with pools of AAV capsid peptides. We used this assay to assess the response of PBMCs from two healthy blood donors to three different peptide pools covering the capsid sequences of AAV2 and AAV9 (FIGS. 12A-12B). In the absence of dasatinib, PBMCs from Donor 1 showed a positive IFN-γ response to AAV9 pool 1 and TNF-α responses to AAV2 pool 2 and AAV9 pools 2 and 3. Donor 2 had positive IFN-γ and/or TNF-α responses to AAV9 pools 2 and 3. All these responses were inhibited in the presence of 100 nM dasatinib.

These results show that dasatinib has the potential to block both aspects of the adaptive immune response to AAV capsid, namely the T cell and antibody responses. This makes this compound promising to efficiently mitigate the immunogenicity of AAV-based gene therapy vectors. Importantly, the inhibitory effect of dasatinib is not restricted to the immune response to the AAV8 serotype, as the T cell responses to AAV2 and AAV9 were also inhibited by dasatinib treatment.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A recombinant viral vector comprising a heterologous polynucleotide for use in the treatment of a disease in an individual, wherein said treatment comprises
(a) the administration of the recombinant viral vector to the individual, and
(b) the administration of a tyrosine kinase inhibitor (TKI) to the individual for prevention or reduction of the formation of anti-drug antibodies (ADAs) related to the administration of the recombinant viral vector.

2. Use of a recombinant viral vector comprising a heterologous polynucleotide in the manufacture of a medicament for the treatment of a disease in an individual, wherein said treatment comprises
(a) the administration of the recombinant viral vector to the individual, and
(b) the administration of a tyrosine kinase inhibitor (TKI) to the individual for prevention or reduction of the formation of anti-drug antibodies (ADAs) related to the administration of the recombinant viral vector.

3. A method for treatment of a disease in an individual, wherein said method comprises
(a) the administration of a recombinant viral vector comprising a heterologous polynucleotide to the individual, and
(b) the administration of a tyrosine kinase inhibitor (TKI) to the individual for prevention or reduction of the formation of anti-drug antibodies (ADAs) related to the administration of the recombinant viral vector.

4. A tyrosine kinase inhibitor (TKI) for use in the prevention or reduction of the formation of anti-drug antibodies (ADAs) related to the administration of a recombinant viral vector comprising a heterologous polynucleotide to an individual.

5. Use of a tyrosine kinase inhibitor (TKI) in the manufacture of a medicament for prevention or reduction of the formation of anti-drug antibodies (ADAs) related to the administration of a recombinant viral vector comprising a heterologous polynucleotide to an individual.

6. A method for preventing or mitigating formation of anti-drug antibodies (ADAs) related to the administration of a recombinant viral vector comprising a heterologous polynucleotide to an individual, comprising the administration of a tyrosine kinase inhibitor (TKI) to the individual.

7. The recombinant viral vector of claim 1, wherein the TKI is a Lck and/or Src kinase inhibitor, particularly dasatinib.

8. The recombinant viral vector of claim 1, wherein (administration of) the TKI causes
(i) inhibition of the formation of ADAs that bind to the recombinant viral vector,
(ii) inhibition of the activation of T cells (induced by the recombinant viral vector),
(iii) inhibition of the cytotoxic activity of T cells (induced by the recombinant viral vector),
(iv) inhibition of activation of B cells (induced by the recombinant viral vector)
(v) inhibition of formation of plasma cells (induced by the recombinant viral vector) and/or
(vi) inhibition of cytokine secretion by immune cells (induced by the recombinant viral vector), particularly wherein said cytokine is one or more cytokine selected from the group consisting of IL-2, TNF-α, IFN-γ, IL-6 and IL-1β.

9. The recombinant viral vector of claim 1, wherein (administration of) the TKI causes reduction of the serum level of one of more cytokine in the individual, particularly wherein said one or more cytokine is selected from the group consisting of IL-2, TNF-α, IFN-γ, IL-6 and IL-1β.

10. The recombinant viral vector of claim 1, wherein administration of the TKI is (i) before, concurrent to, or after the administration of the recombinant viral vector, (ii) intermittently or continuously, and/or (iii) oral.

11 . The recombinant viral vector of claim 1, wherein administration of the TKI is at a dose sufficient to cause
(i) inhibition of the formation of ADAs that bind to the recombinant viral vector,
(ii) inhibition of the activation of T cells (induced by the recombinant viral vector),
(iii) inhibition of the cytotoxic activity of T cells (induced by the recombinant viral vector),
(iv) inhibition of activation of B cells (induced by the recombinant viral vector)
(v) inhibition of formation of plasma cells (induced by the recombinant viral vector) and/or
(vi) inhibition of cytokine secretion by immune cells (induced by the recombinant viral vector), particularly wherein said cytokine is one or more cytokine selected from the group consisting of IL-2, TNF-α, IFN-γ, IL-6 and IL-1β.

12. The recombinant viral vector of claim 1, wherein administration of the TKI is at a dose sufficient to cause reduction of the serum level of one of more cytokine in the individual.

13. The recombinant viral vector of claim 1, wherein administration of the TKI is at a dose sufficient to cause reduction of the secretion of one of more cytokine by immune cells in the individual.

14. The recombinant viral vector of claim 1, wherein administration of the TKI is at an effective dose.

15. The recombinant viral vector of claim 1, wherein administration of the TKI is at a dose of about 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg, particularly at a dose of about 100 mg or lower.

16. The recombinant viral vector of claim 1, wherein administration of the TKI is for the period of time during which the formation of ADAs is expected, and/or is stopped after prevention or reduction of formation of ADAs.

17. The recombinant viral vector of claim 1, wherein administration of the TKI is associated with the first administration of the recombinant viral vector, and optionally is prior, concurrent or subsequent to the first administration of the recombinant viral vector.

18. The recombinant viral vector of claim 1, wherein the administration of the recombinant viral vector is
(i) at an effective dose,
(ii) parenteral, particularly intravenous, and/or
(iii) the first administration of the recombinant viral vector to the individual.

19. The recombinant viral vector of claim 1, one wherein the recombinant viral vector comprises a lentiviral vector, an adenoviral vector or an adeno-associated (AAV) vector.

20. The recombinant viral vector, TKI, use or method of claim 19, wherein the recombinant lentiviral vector comprises envelope proteins to which the ADAs bind.

21. The recombinant viral vector, TKI, use or method of claim 19, wherein the recombinant AAV vector comprises capsid proteins to which the ADAs bind.

22. The recombinant viral vector, TKI, use or method of claim 19, wherein the AA V vector comprises VP1, VP2 and/or VP3 capsid proteins to which the ADAsbind.

23. The recombinant viral vector, TKI, use or method of claim 19, wherein the AAV vector comprises VP1, VP2, and/or VP3 capsid protein having 70% or more sequence identity to VP1, VP2 and/or VP3 capsid protein selected from the group consisting of AAV1, AAV2, AAV3, AAV4, AAV5, AAV6, AAV7, AAV8, AAV9, AAV 10, AAV11, AAV12, -rh74, -rh10, AAV3B, AAV-218 VP1, VP2 and/or VP3 capsid protein.

24. The recombinant viral vector of claim 19, -wherein the AAV vector comprises VP1, VP2, and/or VP3 capsid protein having 100% sequence identity to VP1, VP2 and/or VP3 capsid protein selected from the group consisting of AAV1, AAV2, AAV3, AAV4, AAV5, AAV6, AAV7, AAV8, AAV9, AAV 10, AAV11, AAV12, -rh74, -rh1O, AAV3B, AAV-218 VP1, VP2 and/or VP3 capsid protein.

25. The recombinant viral vector of claim 1, wherein the individual is at risk of developing ADAs that bind to the recombinant viral vector.

26. The recombinant viral vector of claim 1, wherein ADAs that bind to the recombinant viral vector are absent from the individual prior to and/or after administration of the TKI.

27. The recombinant viral vector of claim 1, wherein the individual is at risk of developing ADAs to the polypeptide encoded by said heterologous polynucleotide.

28. The recombinant viral vector of claim 1, wherein the ADAs comprise IgG, IgM, IgA, IgD and/or IgE, in particular wherein the ADAs comprise IgG and/or IgM.

29. The recombinant viral vector of claim 1, wherein the ADAs that bind to the recombinant viral vector are reduced by more than 20%, 30%, 40%, 50%, 60%, 70%, 80%, 90%, 100%, optionally as compared to natural history data of a relevant control group without administration of the TKI.

30. The recombinant viral vector of claim 1, wherein transgene expression is increased upon re-administration of the viral vector, in particular as compared to the transgene expression prior to re-administration of the viral vector.

31. The recombinant viral vector of claim 1, wherein transgene expression is increased by more than 10%, 20%, 30%, 40%, 50%, 60%, 70%, 80%, 90%, or 100% as compared to the transgene expression prior to re-administration of the viral vector.

32. The recombinant viral vector of claim 1, wherein transgene expression is maintained upon re-administration of the viral vector, in particular as compared to the transgene expression prior to re-administration of the viral vector.

33. (canceled)


SEQUENCE LISTING (5 sequences)

SEQ ID NO: 1 - protein, 520 aa, Homo sapiens
MVLGPCMLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGRSRSKRLD

SEQ ID NO: 2 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 3 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 4 - protein, 735 aa, synthetic construct [sequence omitted]

SEQ ID NO: 5 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL